(19) United States
(12) Patent Application Publication
(10) Pub. No.: US 2021/0403557 A1
(43) Pub. Date: Dec. 30, 2021

(54) DOSING REGIMEN OF ANTI-TIGIT ANTIBODY FOR TREATMENT OF CANCER

(71) Applicants: Mingmei CAI, Boston, MA (US); Elliot K. CHARTASH, Rahway, NJ (US); Jane Anne HEALY, Rahway, NJ (US); Mallika LALA, Rahway, NJ (US); Tommy Ruosi LI, Edison, NJ (US); Kapil MAYAWALA, Rahway, NJ (US); Raluca Andreia PREDOIU, Kenilworth, NJ (US); Sybil M.G. WILLIAMS, Boston, MA (US); Zhen ZENG, Westfield, NJ (US); Merck Sharp & Dohme Corp., Rahway, NJ (US)

(72) Inventors: Mingmei Cai, Westwood, MA (US); Elliot K. Chartash, Basking Ridge, NJ (US); Jane Anne Healy, Hoboken, NJ (US); Mallika Lala, West New York, NJ (US); Tommy Ruosi Li, Edison, NJ (US); Kapil Mayawala, Iselin, NJ (US); Raluca Andreia Predoiu, Penn Valley, PA (US); Sybil M.G. Williams, Wayland, MA (US); Zhen Zeng, Westfield, NJ (US)

(73) Assignee: Merck Sharp & Dohme Corp., Rahway, NJ (US)

(21) Appl. No.: 17/288,641

(22) PCT Filed: Nov. 4, 2019
(86) PCT No.: PCT/US2019/059581

§ 371 Date: Apr. 26, 2021

Related U.S. Application Data
(60) Provisional application No. 62/755,805, filed on Nov. 5, 2018.

(51) Int. Cl. C07K 16/28; A61P 35/00

(52) U.S. Cl. CPC C07K 16/2803; A61K 2039/505; A61P 35/00

(57) ABSTRACT

The present invention relates to dosing regimens of an anti-TIGIT antibody useful for the treatment of cancer. In particular, the invention relates to the dosing regimen in a combination therapy which comprises administering an antibody of a Programmed Death 1 protein (PD-1) or Programmed Death Ligand 1 (PD-L1) and an anti-TIGIT antibody.

### RELATED APPLICATIONS

This application claims the benefit of U.S. Provisional Patent Application No. 62/755,805, filed Nov. 5, 2018, which is incorporated by reference herein in its entirety.

### FIELD OF THE INVENTION

The present invention relates to dosing regimens of an anti-TIGIT antibody useful for the treatment of cancer. In particular, the invention relates to the dosing regimen in a combination therapy which comprises administering an antibody directed to a Programmed Death 1 protein (PD-1) or Programmed Death Ligand 1 (PD-L1) and an anti-TIGIT (T cell immunoreceptor with Ig and ITIM domains) antibody.

### BACKGROUND OF THE INVENTION

PD-1 is recognized as an important molecule in immune regulation and the maintenance of peripheral tolerance. PD-1 is moderately expressed on naive T, B and NKT cells and up-regulated by T/B cell receptor signaling on lymphocytes, monocyte and myeloid cells (1).

Two known ligands for PD-1, PD-L1 (B7-H1) and PD-L2 (B7-DC), are expressed in human cancers arising in various tissues. In large sample sets of e.g. ovarian, renal, colorectal, pancreatic, liver cancers and melanoma, it was shown that PD-L 1 expression correlated with poor prognosis and reduced overall survival irrespective of subsequent treatment (2-13). Similarly, PD-1 expression on tumor infiltrating lymphocytes was found to mark dysfunctional T cells in breast cancer and melanoma (14-15) and to correlate with poor prognosis in renal cancer (16). Thus, it has been proposed that PD-L1 expressing tumor cells interact with PD-1 expressing T cells to attenuate T cell activation and evasion of immune surveillance, thereby contributing to an impaired immune response against the tumor. Several monoclonal antibodies that inhibit the interaction between PD-1 and one or both of its ligands PD-L1 and PD-L2 have been approved for treating cancer. Pembrolizumab is a potent humanized immunoglobulin G4 (IgG4) mAb with high specificity of binding to the programmed cell death 1 (PD 1) receptor, thus inhibiting its interaction with programmed cell death ligand 1 (PD-L1) and programmed cell death ligand 2 (PD-L2). Based on preclinical in vitro data, pembrolizumab has high affinity and potent receptor blocking activity for PD-1. Keytruda® (pembrolizumab) is indicated for the treatment of patients across a number of indications.

TIGIT (T cell immunoreceptor with Ig and ITIM domains) is an immunomodulatory receptor expressed primarily on activated T cells and NK cells. TIGIT is also known as VSIG9; VSTM3; and WUCAM. Its structure shows one extracellular immunoglobulin domain, a type 1transmembrane region and two ITIM motifs. TIGIT forms part of a co-stimulatory network that consists of positive (CD226) and negative (TIGIT) immunomodulatory receptors on T cells, and ligands expressed on APCs (CD155 and CD112). An important feature in the structure of TIGIT is the presence of an immunoreceptor tyrosine-based inhibition motif (ITIM) in its cytoplasmic tail domain. As with PD-1 and CTLA-4, the ITIM domain in the cytoplasmic region of TIGIT is predicted to recruit tyrosine phosphatases, such as SHP-1 and SHP-2, and subsequent de-phosphorylation of tyrosine residues with in the immunoreceptor tyrosine-base activation motifs (ITAM) on T cell receptor (TCR) subunits. Hence, ligation of TIGIT by receptor-ligands CD155 and CD112 expressed by tumor cells or TAMS may contribute to the suppression of TCR-signaling and T cell activation, which is essential for mounting effective anti-tumor immunity. Thus, an antagonist antibody specific for TIGIT could inhibit the CD155 and CD112 induced suppression of T cell responses and enhance anti-tumor immunity. Anti-TIGIT antibodies have been described in WO2016/028656 and WO2017/030823.

Selecting a dosage regimen for an anti-TIGIT antibody monotherapy or combination therapy with anti-PD-1 or anti-PD-L1 therapy depends on several factors, including the serum or tissue turnover rate of the entity, the level of symptoms, the immunogenicity of the entity, antidrug antibody endpoints and the accessibility of the target cells, tissue or organ in the individual being treated, as well as safety. Formation of antidrug antibodies can potentially confound drug exposures at therapeutic doses, and prime for subsequent infusion-related toxicities. In addition, anti-TIGIT and/or anti-PD-1/anti-PD-L1 treatment can result in immune stimulation and the potential for cytokine release that affects safety.

### SUMMARY OF THE INVENTION

The invention provides a method for treating cancer in a patient comprising administering 2.1 mg-700 mg of an anti-TIGIT antibody 31C6 or 31C6 variant. In one embodiment, the method optionally comprises co-administration with an anti-PD-1 or anti-PD-L1 antibody. In one embodiment, the anti-TIGIT antibody and anti-PD-1 antibody are co-formulated. In a further embodiment, the tumor cells of the individual are PD-L1 expression positive. In one embodiment, the anti-PD-1 antibody blocks the binding of PD-1 to PD-L1 and PD-L2. The invention also provides a pharmaceutical composition comprising 2.1 mg to 700 mg of anti-TIGIT antibody 31C6 or a 31C6 variant, and 200 mg of pembrolizumab or pembrolizumab variant.

An aspect of the invention provides a method for treating cancer in a patient comprising administering to the patient 2.1 mg to 700 mg of an anti-TIGIT antibody comprising a heavy chain and a light chain, wherein the light chain comprises light chain CDRs of SEQ ID NOs: 26, 27 and 28 and the heavy chain comprises heavy chain CDRs of SEQ ID NOs: 29, 30 and 31.

In various embodiments of the method, the anti-TIGIT antibody is administered via intravenous infusion.

In various embodiments of the method, the patient is administered about 2.1 mg to about 700 mg of the anti-TIGIT antibody. In various embodiments of the method, the patient is administered about 2.1 mg of the anti-TIGIT antibody. In various embodiments of the method, the patient is administered about 7 mg of the anti-TIGIT antibody. In various embodiments of the method, the patient is administered about 21 mg of the anti-TIGIT antibody. In various embodiments of the method, the patient is administered about 70 mg of the anti-TIGIT antibody. In various embodiments of the method, the patient is administered about 200 mg of the anti-TIGIT antibody. In various embodiments of the method, the patient is administered about 210 mg of the anti-TIGIT antibody. In various embodiments of the method, the patient is administered about 700 mg of the anti-TIGIT antibody.

In various embodiments of the method, the patient is administered 2.1 mg of the anti-TIGIT antibody. In various embodiments of the method, the patient is administered 7 mg of the anti-TIGIT antibody. In various embodiments of the method, the patient is administered 21 mg of the anti-TIGIT antibody. In various embodiments of the method, the patient is administered 70 mg of the anti-TIGIT antibody. In various embodiments of the method, the patient is administered 200 mg of the anti-TIGIT antibody. In various embodiments of the method, the patient is administered 210 mg of the anti-TIGIT antibody. In various embodiments of the method, the patient is administered 700 mg of the anti-TIGIT antibody.

In various embodiments of the method, the patient is administered the anti-TIGIT antibody on Day 1 and then once every three weeks thereafter. For example, the duration of the treatment is weeks or months.

In various embodiments of the method, the anti-TIGIT antibody comprises a heavy chain and a light chain, and wherein the heavy chain comprises a heavy chain variable region comprising SEQ ID NO:25. In various embodiments of the method, the anti-TIGIT antibody comprises a heavy chain and a light chain, and the light chain comprises a light chain variable region comprising SEQ ID NO: 24. In various embodiments of the method, the anti-TIGIT antibody comprises a heavy chain and a light chain, and wherein the heavy chain comprises a heavy chain variable region comprising SEQ ID NO:25 and the light chain comprises a light chain variable region comprising SEQ ID NO: 24.

In various embodiments of the method, the anti-TIGIT antibody comprises a heavy chain and a light chain, and wherein the heavy chain comprises SEQ ID NO:23. In various embodiments of the method, the anti-TIGIT antibody comprises a heavy chain and a light chain, and the light chain comprises SEQ ID NO:22. In various embodiments of the method, the anti-TIGIT antibody comprises a heavy chain and a light chain, and wherein the heavy chain comprises SEQ ID NO:23 and the light chain comprises SEQ ID NO:22.

In various embodiments of the method, the anti-TIGIT antibody is a 31C6 variant.

In various embodiments of the method, the 31C6 variant comprises a heavy chain and a light chain, and wherein the heavy chain comprises a heavy chain variable region comprising 80%, 85%, 90%, 95%, or 99% sequence identity to SEQ ID NO:25. In various embodiments of the method, the 31C6 variant comprises a heavy chain and a light chain, and the light chain comprises a light chain variable region comprising 80%, 85%, 90%, 95%, or 99% sequence identity to SEQ ID NO: 24. In various embodiments of the method, the 31C6 variant comprises a heavy chain and a light chain, and wherein the heavy chain comprises a heavy chain variable region comprising 80%, 85%, 90%, 95%, or 99% sequence identity to SEQ ID NO:25 and the light chain comprises a light chain variable region comprising 80%, 85%, 90%, 95%, or 99% sequence identity to SEQ ID NO: 24.

In various embodiments of the method, the 31C6 variant comprises a heavy chain and a light chain, and wherein the heavy chain comprises 80%, 85%, 90%, 95%, or 99% sequence identity to SEQ ID NO:23. In various embodiments of the method, the 31C6 variant comprises a heavy chain and a light chain, and the light chain comprises 80%, 85%, 90%, 95%, or 99% sequence identity to SEQ ID NO:22. In various embodiments of the method, the 31C6 variant comprises a heavy chain and a light chain, and wherein the heavy chain comprises 80%, 85%, 90%, 95%, or 99% sequence identity to SEQ ID NO:23 and the light chain comprises 80%, 85%, 90%, 95%, or 99% sequence identity to SEQ ID NO:22.

In various embodiments of the method, the anti-TIGIT antibody is co-administered with an anti-PD-1 antibody or anti-PD-L1 antibody, or antigen binding fragment thereof.

In various embodiments of the method, the anti-TIGIT antibody is co-formulated with an anti-PD-1 antibody or anti-PD-L1 antibody or antigen binding fragment thereof.

In various embodiments of the method, the anti-PD-1 antibody, or antigen binding fragment thereof, specifically binds to human PD-1 and blocks the binding of human PD-L1 to human PD-1.

In various embodiments of the method, the anti-PD-1 antibody, or antigen binding fragment thereof, also blocks binding of human PD-L2 to human PD-1.

In various embodiments of the method, the anti-PD-1 antibody, or antigen binding fragment thereof comprises: (a) light chain CDRs of SEQ ID NOs: 1, 2 and 3 and (b) heavy chain CDRs of SEQ ID NOs: 6, 7 and 8. In various embodiments of the method, the anti-PD-1 antibody is a 31C6 variant that comprises: (a) light chain CDRs of SEQ ID NOs: 1, 2 and 3 and (b) heavy chain CDRs of SEQ ID NOs: 6, 7 and 8.

In various embodiments of the method, the anti-PD-1 antibody comprises a heavy chain and a light chain, and wherein the heavy chain comprises a heavy chain variable region comprising SEQ ID NO:9 and the light chain comprises a light chain variable region comprising SEQ ID NO: 4.

In various embodiments of the method, the anti-PD-1 antibody comprises a heavy chain and a light chain, and wherein the heavy chain comprises SEQ ID NO:10 and the light chain comprises SEQ ID NO:5. For example, the anti-PD-1 antibody is pembrolizumab. In various embodiments of the method, the anti-PD-1 antibody is a pembrolizumab variant. In various embodiments of the method, the anti-PD-1 antibody is nivolumab. In various embodiments of the method, the anti-PD-L1 antibody is atezolizumab, durvalumab, or avelumab. In various embodiments of the method, the anti-PD-1 antibody is administered at 200 mg via intravenous infusion on Day 1 and then once every three weeks thereafter. In various embodiments of the method, the anti-PD-1 antibody is administered at 400 mg via intravenous infusion on Day 1 and then once every six weeks thereafter.

In various embodiments of the method, the anti-PD-1 antibody is a humanized anti-PD-1 antibody that comprises a heavy chain and a light chain, and wherein the heavy chain comprises a heavy chain variable region comprising heavy chain CDRs of SEQ ID NOs: 6, 7 and 8 and the light chain comprises a light chain variable region comprising light chain CDRs of SEQ ID NOs: 1, 2 and 3; and the anti-TIGIT antibody is a humanized anti-TIGIT antibody which comprises a heavy chain and a light chain, and wherein the heavy chain comprises a heavy chain variable region comprising heavy chain CDRs of SEQ ID NOs: 29, 30 and 31 and the light chain comprises a light chain variable region comprising light chain CDRs of SEQ ID NOs: 26, 27 and 28.

In various embodiments of the method, the anti-PD-1 antibody comprises a heavy chain and a light chain, and wherein the heavy chain comprises a heavy chain variable region comprising SEQ ID NO:9 and the light chain comprises a light chain variable region comprising SEQ ID NO: 4; and the anti-TIGIT antibody comprises a heavy chain and a light chain, and wherein the heavy chain comprises a heavy chain variable region comprising SEQ ID NO:25 and the light chain comprises a light chain variable region comprising SEQ ID NO: 24.

In various embodiments of the method, the anti-PD-1 antibody comprises a heavy chain and a light chain, and wherein the heavy chain comprises SEQ ID NO:10 and the light chain comprises SEQ ID NO: 5; and the anti-TIGIT antibody comprises a heavy chain and a light chain, and wherein the heavy chain comprises SEQ ID NO:23 and the light chain comprises a light chain variable region comprising SEQ ID NO: 22.

In various embodiments of the method, the anti-PD-1 antibody is administered at 200 mg via intravenous infusion on Day 1 and then once every three weeks thereafter, and the anti-TIGIT antibody is administered at 200 mg via intravenous infusion on Day 1 and then once every three weeks thereafter.

In various embodiments of the method, the anti-PD-1 antibody is administered at 400 mg via intravenous infusion on Day 1 and then once every six weeks thereafter, and the anti-TIGIT antibody is administered at 200 mg via intravenous infusion on Day 1 once every three weeks.

In various embodiments of the method, the anti-PD-1 antibody is administered at 200 mg via intravenous infusion on Day 1 and then once every three weeks thereafter, and the anti-TIGIT antibody is administered at 700 mg via intravenous infusion on Day 1 and then once every three weeks thereafter.

In various embodiments of the method, the anti-PD-1 antibody is administered at 400 mg via intravenous infusion on Day 1 and then once every six weeks thereafter, and the anti-TIGIT antibody is administered at 700 mg via intravenous infusion on Day 1 once every three weeks.

In various embodiments of the method, 200 mg of anti-PD-1 antibody is co-formulated with 2.1 mg to 700 mg anti-TIGIT antibody. For example, 200 mg of anti-PD-1 antibody is co-formulated with 200 mg anti-TIGIT antibody.

In various embodiments of the method, 200 mg of anti-PD-1 antibody is co-formulated with an amount of anti-TIGIT antibody described in the Examples. In various embodiments of the method, 200 mg of anti-PD-1 antibody is co-formulated with 7 mg anti-TIGIT antibody. In various embodiments of the method, 200 mg of anti-PD-1 antibody is co-formulated with 21 mg anti-TIGIT antibody. In various embodiments of the method, 200 mg of anti-PD-1 antibody is co-formulated with 70 mg anti-TIGIT antibody. In various embodiments of the method, 200 mg of anti-PD-1 antibody is co-formulated with 210 mg anti-TIGIT antibody. In various embodiments of the method, 200 mg of anti-PD-1 antibody is co-formulated with 200 mg anti-TIGIT antibody. In various embodiments of the method, 200 mg of anti-PD-1 antibody is co-formulated with 700 mg anti-TIGIT antibody.

In various embodiments of the method, the cancer is selected from a cancer disclosed in Part A or Part B described in the Examples. In various embodiments of the method, the cancer is at least one from the group consisting of: NSCLC, colorectal cancer, cervical cancer, gastric cancer, breast cancer, ovarian, epithelial, fallopian tube, or primary peritoneal carcinoma. For example, the cancer is NSCLC. In various embodiments, the subject or patient has a cancer and expresses at least one Breast Cancer gene (e.g., BRCA). In various embodiments, the cancer or a sample from the subject is found to have a level or to express at least one Breast Cancer gene (BRCA). In various embodiments, the at least one BRCA gene is BRCA1 or BRCA2. In an embodiment, the cancer is BRCA negative. For example, the cancer (for example breast cancer and ovarian cancer) is a BRCA negative cancer. In an embodiment, the cancer is BRCA positive.

In various embodiments, the method further comprises administering a combination of (i) carboplatin and pemetrexed (5-substituted pyrrolo[2,3-d]pyrimidine) or (ii) carboplatin and paclitaxel.

In various embodiments of the method, the individual has not been previously treated with anti-PD-1 or anti-PD-L1 therapy or is confirmed progressive while receiving prior anti-PD-1 or anti-PD-L1 therapy.

An aspect of the invention provides a pharmaceutical composition comprising 200 mg pembrolizumab or a pembrolizumab variant, an anti-TIGIT 31C6 antibody or antigen binding fragment or 31C6 variant, and a pharmaceutically acceptable excipient. In various embodiments, the pharmaceutical composition comprises about 2.1 mg to about 700 mg of 31C6 antibody or antigen binding fragment or a 31C6 variant. In various embodiments, the 31C6 antibody comprises 2.1 mg to 700 mg of anti-TIGIT antibody 31C6 antibody or antigen binding fragment or a 31C6 variant.

In various embodiments, the pharmaceutical composition comprises 2.1 mg of the anti-TIGIT antibody. In various embodiments, the pharmaceutical composition comprises 7 mg of the anti-TIGIT 31C6 antibody or antigen binding fragment or 31C6 variant. In various embodiments, the pharmaceutical composition comprises 21 mg of the anti-TIGIT 31C6 antibody or antigen binding fragment or 31C6 variant. In various embodiments, the pharmaceutical composition comprises 70 mg of the anti-TIGIT 31C6 antibody or antigen binding fragment or 31C6 variant. In various embodiments, the pharmaceutical composition comprises 200 mg of the anti-TIGIT 31C6 antibody or antigen binding fragment or 31C6 variant. In various embodiments of the method, the pharmaceutical composition comprises 210 mg of the anti-TIGIT 31C6 antibody or antigen binding fragment or 31C6 variant. In various embodiments of the method, the pharmaceutical composition comprises 700 mg of the anti-TIGIT 31C6 antibody or antigen binding fragment or 31C6 variant.

An aspect of the invention provides a pharmaceutical composition comprising 200 mg pembrolizumab or a pembrolizumab variant, 200 mg of 31C6 antibody or a 31C6 variant, and a pharmaceutically acceptable excipient.

In various embodiments of the method, the 31C6 antibody comprises a heavy chain and a light chain, and wherein the heavy chain comprises a heavy chain variable region comprising SEQ ID NO:25. In various embodiments, the 31C6 antibody comprises a heavy chain and a light chain, and the light chain comprises a light chain variable region comprising SEQ ID NO: 24. In various embodiments, the 31C6 antibody comprises a heavy chain and a light chain, and wherein the heavy chain comprises a heavy chain variable region comprising SEQ ID NO:25 and the light chain comprises a light chain variable region comprising SEQ ID NO: 24.

In various embodiments, the 31C6 antibody comprises a heavy chain and a light chain, and wherein the heavy chain comprises SEQ ID NO:23. In various embodiments, the 31C6 antibody comprises a heavy chain and a light chain, and the light chain comprises SEQ ID NO:22. In various embodiments, the anti-TIGIT antibody comprises a heavy chain and a light chain, and wherein the heavy chain comprises SEQ ID NO:23 and the light chain comprises SEQ ID NO:22.

In various embodiments, the 31C6 antibody is a 31C6 variant.

In various embodiments, the 31C6 variant comprises a heavy chain and a light chain, and wherein the heavy chain comprises a heavy chain variable region comprising 80%, 85%, 90%, 95%, or 99% sequence identity to SEQ ID NO:25. In various embodiments, the 31C6 variant comprises a heavy chain and a light chain, and the light chain comprises a light chain variable region comprising 80%, 85%, 90%, 95%, or 99% sequence identity to SEQ ID NO: 24. In various embodiments, the 31C6 variant comprises a heavy chain and a light chain, and wherein the heavy chain comprises a heavy chain variable region comprising 80%, 85%, 90%, 95%, or 99% sequence identity to SEQ ID NO:25 and the light chain comprises a light chain variable region comprising 80%, 85%, 90%, 95%, or 99% sequence identity to SEQ ID NO: 24.

In various embodiments, the 31C6 variant comprises a heavy chain and a light chain, and wherein the heavy chain comprises 80%, 85%, 90%, 95%, or 99% sequence identity to SEQ ID NO:23. In various embodiments, the 31C6 variant comprises a heavy chain and a light chain, and the light chain comprises 80%, 85%, 90%, 95%, or 99% sequence identity to SEQ ID NO:22. In various embodiments, the 31C6 variant comprises a heavy chain and a light chain, and wherein the heavy chain comprises 80%, 85%, 90%, 95%, or 99% sequence identity to SEQ ID NO:23 and the light chain comprises 80%, 85%, 90%, 95%, or 99% sequence identity to SEQ ID NO:22.

In various embodiments, the anti-TIGIT antibody is formulated to be co-administered with pembrolizumab or a pembrolizumab variant.

In various embodiments, the anti-TIGIT antibody is co-formulated with pembrolizumab or a pembrolizumab variant.

In various embodiments, the pembrolizumab or a pembrolizumab variant thereof comprises: (a) light chain CDRs of SEQ ID NOs: 1, 2 and 3 and (b) heavy chain CDRs of SEQ ID NOs: 6, 7 and 8. In various embodiments, the anti-PD-1 antibody is a 31C6 variant that comprises: (a) light chain CDRs of SEQ ID NOs: 1, 2 and 3 and (b) heavy chainCDRs of SEQ ID NOs: 6, 7 and 8.

In various embodiments, the pembrolizumab or a pembrolizumab variant comprises a heavy chain and a light chain, and wherein the heavy chain comprises a heavy chain variable region comprising SEQ ID NO:9 and the light chain comprises a light chain variable region comprising SEQ ID NO: 4.

In various embodiments, the pembrolizumab or a pembrolizumab variant comprises a heavy chain and a light chain, and wherein the heavy chain comprises SEQ ID NO:10 and the light chain comprises SEQ ID NO:5. For example, the anti-PD-1 antibody is pembrolizumab. In various embodiments, the anti-PD-1 antibody is a pembrolizumab variant. In various embodiments, the anti-PD-1 antibody is nivolumab.
In various embodiments, the pembrolizumab or a pembrolizumab variant is formulated for intravenous infusion.

In various embodiments, the pembrolizumab or a pembrolizumab variant comprises a heavy chain and a light chain, and wherein the heavy chain comprises a heavy chain variable region comprising heavy chain CDRs of SEQ ID NOs: 6, 7 and 8 and the light chain comprises a light chain variable region comprising light chain CDRs of SEQ ID NOs: 1, 2 and 3; and the a 31C6 antibody is a humanized anti-TIGIT antibody which comprises a heavy chain and a light chain, and wherein the heavy chain comprises a heavy chain variable region comprising heavy chain CDRs of SEQ ID NOs: 29, 30 and 31 and the light chain comprises a light chain variable region comprising light chain CDRs of SEQ ID NOs: 26, 27 and 28.

In various embodiments, the pembrolizumab or a pembrolizumab variant comprises a heavy chain and a light chain, and wherein the heavy chain comprises a heavy chain variable region comprising SEQ ID NO:9 and the light chain comprises a light chain variable region comprising SEQ ID NO: 4; and the 31C6 antibody comprises a heavy chain and a light chain, and wherein the heavy chain comprises a heavy chain variable region comprising SEQ ID NO:25 and the light chain comprises a light chain variable region comprising SEQ ID NO: 24.

In various embodiments, the pembrolizumab or a pembrolizumab variant comprises a heavy chain and a light chain, and wherein the heavy chain comprises SEQ ID NO:10 and the light chain comprises SEQ ID NO: 5; and the anti-TIGIT antibody comprises a heavy chain and a light chain, and wherein the heavy chain comprises SEQ ID NO:23 and the light chain comprises a light chain variable region comprising SEQ ID NO: 22. An aspect of the invention provides a pharmaceutical composition comprising 200 mg pembrolizumab or a pembrolizumab variant, 2.1 to 700 mg of 31C6 antibody or a 31C6 variant, and a pharmaceutically acceptable excipient.

An aspect of the invention provides a pharmaceutical composition comprising 400 mg pembrolizumab or a pembrolizumab variant, 2.1 to 700 mg of 31C6 antibody or a 31C6 variant, and a pharmaceutically acceptable excipient.

In various embodiments, the pharmaceutical composition comprises 2.1 mg of the 31C6 antibody or 31C6 variant. In various embodiments, the pharmaceutical composition comprises 7 mg of the 31C6 antibody or 31C6 variant. In various embodiments, the pharmaceutical composition comprises 21 mg of the 31C6 antibody or 31C6 variant. In various embodiments, the pharmaceutical composition comprises 70 mg of the 31C6 antibody or 31C6 variant. In various embodiments, the pharmaceutical composition comprises 200 mg of the 31C6 antibody or 31C6 variant. In various embodiments, the pharmaceutical composition comprises 210 mg of the 31C6 antibody or 31C6 variant. In various embodiments, the pharmaceutical composition comprises 700 mg of the 31C6 antibody or 31C6 variant.

In various embodiments of the pharmaceutical composition, the 31C6 antibody or 31C6 variant comprises a heavy chain and a light chain, wherein the light chain comprises light chain CDRs of SEQ ID NOs: 26, 27 and 28 and the heavy chain comprises heavy chain CDRs of SEQ ID NOs: 29, 30 and 31.

In various embodiments of the pharmaceutical composition, the 31C6 antibody comprises a heavy chain and a light chain, wherein the heavy chain comprises a heavy chain variable region comprising SEQ ID NO:25 and the light chain comprises a light chain variable region comprising SEQ ID NO: 24.

In various embodiments of the pharmaceutical composition, the 31C6 antibody comprises a heavy chain and a light chain, and wherein the heavy chain comprises SEQ ID NO:23 and the light chain comprises a light chain variable region comprising SEQ ID NO: 22.

An aspect of the invention provides a kit for treating cancer comprising any of the pharmaceutical compositions described herein. In various embodiments, the composition further comprises instructions for use.

### BRIEF DESCRIPTION OF THE DRAWINGS

FIG. 1 A plot generated pooling 31C6 anti-TIGIT antibody pharmacokinetic data from both the monotherapy and the combination therapy arms. Arithmetic mean concentrations plotted using nominal sampling times.

FIGS. 2A and 2B Each show a waterfall plot of subjects with best percentage change from baseline in target lesions based on investigator assessment per RECIST v1.1 (evaluated in patients with measurable disease at baseline and >1 evaluable post-baseline imaging assessment (n=25 for 31C6 monotherapy and n=41 for 31C6 combination therapy with pembrolizumab)). Each bar represents an individual subject. The data for the combination therapy includes 32 patients originally allocated to the combination and 9 who crossed over from monotherapy. For the 31C6 monotherapy, 25% showed any decrease and 4% showed a decrease≥−30%. For the 31C6 and pembrolizumab combination therapy, 32% showed any decrease and 24% showed a decrease≥−30%.

FIGS. 3A and 3B Each show a plot of subjects showing treatment duration and response based on investigator assessment per RECIST v1.1. Line length represents the time to the last dose of study treatment. Time to best response and subsequent PD or death, whichever occurred first, are shown for each patient. Only those patients who had ≥1 post-baseline imaging assessment are included. * represent those patients who crossed over from monotherapy to combination therapy. The combination therapy includes 32 patients who originally allocated to the combination therapy and 9 who crossed over from the 31C6 monotherapy.

FIG. 4 shows a plot of PD-1 naïve NSCLC patients showing treatment duration and response based on investigator assessment per RECIST v1.1.

FIGS. 5A and 5B show plots of PD-1 refractory NSCLC patients showing treatment duration and response based on investigator assessment per RECIST v1.1. Patients were either administered 200 mg 31C6 antibody monotherapy (FIG. 5A) or a combination therapy of 200 mg 31C6 antibody and 200 mg pembrolizumab (FIG. 5B) as described in Example 2.

FIG. 6 shows a plot of refractory ovarian patients showing treatment duration and response based on investigator assessment per RECIST v1.1. Data compiles results for treatment as described in Part A (31C6 monotherapy) and Part B (for 31C6 combination therapy with pembrolizumab) of Example 1.

FIG. 7 shows a plot of PD-1 naïve breast cancer patients showing treatment duration and response based on investigator assessment per RECIST v1.1. Patients were treated with a 200 mg 31C6 combination therapy with pembrolizumab (200 mg) as described in Example 2.

FIG. 8 shows a plot of PD-1 naïve CRC breast cancer patients showing treatment duration and response based on investigator assessment per RECIST v1.1. Patients were treated with a 200 mg 31C6 combination therapy with pembrolizumab (200 mg) as described in Example 2.

FIGS. 9A and 9B show plots of cervical patients showing treatment duration and response based on investigator assessment per RECIST v1.1. Patients were either administered 200 mg (FIG. 9A) or 700 mg (FIG. 9B) of 31C6 antibody in a combination therapy with 200 mg pembrolizumab as described in Example 2.

### DETAILED DESCRIPTION OF THE INVENTION

Abbreviations. Throughout the detailed description and examples of the invention the following abbreviations will be used:
ADA Anti-drug antibody
AE Adverse event
ALT Alanine aminotransferase
ANC Absolute neutrophil count
aPTT Activated partial thromboplastin time
ASaT All-Subjects-as-Treated
ASCO American Society of Clinical Oncology
AST Aspartate aminotransferase
AUC Area under the curve
BCG Bacillus Calmette-Guérin
β-hCG β-human chorionic gonadotropin
BOR Best overall response
BID One dose twice daily
CBR Clinical Benefit Rate
CDR Complementarity determining region
CHO Chinese hamster ovary
Cₘₐₓ Maximum concentration
Cₘᵢₙ Minimum concentration
CNS Central nervous system
CR Complete Response
CRF Case report form
CSF Colony-stimulating factor
CSR Clinical Study Report
CT Computed tomography
CTCAE Common Terminology Criteria for Adverse Events
CTLA-4 Cytotoxic T lymphocyte-associated antigen 4
D De-escalate to the next lower dose
DCR Disease Control Rate
DFS Disease free survival
DILI Drug-induced liver injury
DLT Dose limiting toxicity
DNA Deoxyribonucleic acid
DOR Duration of response
DSDR Durable Stable Disease Rate
DU Unacceptably toxic dose
ECG Electrocardiogram
ECI Event(s) of clinical interest
ECOG Eastern Cooperative Oncology Group
eCRF Electronic case report form
EMA European Medicines Agency
FAS Full Analysis Set
ELISA Enzyme-linked immunoassay
FBR Future biomedical research
FFPE Formalin-fixed, paraffin-embedded
FR Framework region
FSH Follicle-stimulating hormone
G3 CTCAE (adverse event grading scale) grade 3
GCP Good Clinical Practice
GFR Glomerular filtration rate
GGT Gamma glutamyl transferase
HBsAg Hepatitis B surface antigen
HCV Hepatitis C virus
HIV Human immunodeficiency virus
HRT Hormonal replacement therapy
IB Investigator's Brochure
ICF Informed consent form
ICH International Conference on Harmonisation
iCPD iRECIST confirmed progressive disease
IEC Independent Ethics Committee
Ig Immunoglobulin
IgG Immunoglobulin G
IHC Immunohistochemistry or immunohistochemical
IL Interleukin
IM Intramuscular
INR International normalized ratio
IO Immune oncology agent
irAE Immune-related adverse event
IRB Institutional Review Board
iRECIST Modified RECIST 1.1 for immune-based herapeutics
irRC Immune related response criteria
iSD iRECIST stable disease
iUPD iRECIST unconfirmed progressive disease
IV Intravenous
IVRS Interactive Voice Response System
IWRS Integrated Web Response System
LDH Lactate dehydrogenase
LFT Liver function tests
mAb Monoclonal antibody
MASCC Multinational Association of Supportive Care in Cancer
MRI Magnetic resonance imaging
mRNA Messenger ribonucleic acid
MSD Merck Sharp & Dohme Corp., a subsidiary of Merck & Co., Inc.
MSI Microsatellite instability
MTD Maximum tolerated dose
mTPI Modified toxicity probability interval
NCBI National Center for Biotechnology Information
NCI National Cancer Institute
NK Natural killer
NOAEL No observed adverse effect level
NSAID Non-steroidal anti-inflammatory drug
NSCLC Non-small cell lung cancer
ORR Objective response rate
OS Overall survival
OTC Over-the-counter
PBMC Peripheral blood mononuclear cell
PD Progressive disease
PD-1 Programmed Death 1
PD-L1 Programmed Cell Death 1 Ligand 1
PD-L2 Programmed Cell Death 1 Ligand 2
PFS Progression free survival
PK Pharmacokinetic(s)
PNA Pneumonia
PP Per-Protocol
PR Partial response
PT Prothrombin time
PTT Partial thromboplastin time
Q2W One dose every two weeks
Q3W One dose every three weeks
QD One dose per day
RECIST Response Evaluation Criteria in Solid Tumors
RECIST Response Evaluation Criteria in Solid Tumors,
1.1 version 1.1
RNA Ribonucleic acid
RP2D Recommended Phase 2 dose
SAE Serious adverse event
SAP Statistical Analysis Plan
SD Stable disease
SGOT Serum glutamic oxaloacetic transaminase
SGPT Serum glutamic pyruvic transaminase
SNP Single nucleotide polymorphism
SoA Schedule of Activities
sSAP Supplementary Statistical Analysis Plan
TCR T cell receptor
TIGIT T cell immunoreceptor with Ig and ITIM domains
TNBC Triple-negative breast cancer
TNF Tumor necrosis factor
TSH Thyroid-stimulating hormone
ULN Upper limit of normal
VH Immunoglobulin heavy chain variable region
VK Immunoglobulin kappa light chain variable region
WOCBP Woman of childbearing potential

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

As used herein, an “31C6 variant” means a monoclonal antibody which comprises heavy chain and light chain sequences that are substantially identical to those in 31C6 (as described below and in WO2016/028656, incorporated by reference in its entirety), except for having three, two or one conservative amino acid substitutions at positions that are located outside of the light chain CDRs and six, five, four, three, two or one conservative amino acid substitutions that are located outside of the heavy chain CDRs, e.g., the variant positions are located in the FR regions or the constant region, and optionally has a deletion of the C-terminal lysine residue of the heavy chain. In other words, 31C6 and a 31C6 variant comprise identical CDR sequences, but differ from each other due to having a conservative amino acid substitution at no more than three or six other positions in their full length light and heavy chain sequences, respectively. A 31C6 variant is substantially the same as 31C6 with respect to the following properties: binding affinity to human TIGIT and ability to block the binding of human TIGIT to human CD155 and human CD112.

“Administration” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biologicalfluid. Treatment of a cell encompasses contact of a reagent to the cell, as well as contact of a reagent to a fluid, where the fluid is in contact with the cell. The term “subject” includes any organism, preferably an animal, more preferably a mammal (e.g., rat, mouse, dog, cat, rabbit) and most preferably a human.

As used herein, the term “antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, chimeric antibodies and camelized single domain antibodies. “Parental antibodies” are antibodies obtained by exposure of an immune system to an antigen prior to modification of the antibodies for an intended use, such as humanization of an antibody for use as a human therapeutic.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

As used herein, unless otherwise indicated, “antibody fragment” or “antigen binding fragment” refers to antigen binding fragments of antibodies, i.e. antibody fragments that retain the ability to bind specifically to the antigen bound by the full-length antibody, e.g. fragments that retain one or more CDR regions. Examples of antibody binding fragments include, but are not limited to, Fab, Fab′, F(ab′)₂, and Fv fragments; diabodies; linear antibodies; single-chain antibody molecules, e.g., sc-Fv; nanobodies and multispecific antibodies formed from antibody fragments.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g. without producing undesired results such as false positives. Antibodies, or binding fragments thereof, useful in the present invention will bind to the target protein with an affinity that is at least two fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, e.g. the amino acid sequence of a mature human PD-1 or human PD-L1 molecule, if it binds to polypeptides comprising that sequence but does not bind to proteins lacking that sequence.

“Carboplatin” as used herein refers to a second-generation platinum compound with a broad spectrum of antineoplastic properties. See U.S. Pat. Nos. 10,421,770, 8,377,888, and 6,770,653. Carboplatin contains a platinum atom complexed with two ammonia groups and a cyclobutane-dicarboxyl residue. This agent is activated intracellularly to form reactive platinum complexes that bind to nucleophilic groups such as GC-rich sites in DNA, thereby inducing intrastrand and interstrand DNA cross-links, as well as DNA-protein cross-links. These carboplatin-induced DNA and protein effects result in apoptosis and cell growth inhibition. This agent possesses tumoricidal activity similar to that of its parent compound, cisplatin, but is more stable and less toxic. Carboplatin analogs may also be administered for cancer treatment. See U.S. Pat. No. 6,548,541B1.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Co-administration” as used herein for agents such as the PD-1 antagonist or TIGIT antagonist means that the agents are administered so as to have overlapping therapeutic activities, and not necessarily that the agents are administered simultaneously to the subject. The agents may or may not be in physical combination prior to administration. In an embodiment, the agents are administered to a subject simultaneously or at about the same time. For example, the anti-PD-1 antibody and anti-TIGIT antibody drug products contained in separate vials, when in liquid solution, may be mixed into the same intravenous infusion bag or injection device, and administered simultaneously to the patient.

“Co-formulated” or “co-formulation” or “coformulation” or “coformulated” as used herein refers to at least two different antibodies or antigen binding fragments thereof which are formulated together and stored as a combined product in a single vial or vessel (for example an injection device) rather than being formulated and stored individually and then mixed before administration or separately administered. In one embodiment, the co-formulation contains two different antibodies or antigen binding fragments thereof.

Pharmacokinetic “steady state” is a period of time during which any accumulation of drug concentrations owing to multiple doses has been maximized and systemic drug exposure is considered uniform after each subsequent dose administered; in the specific case of pembrolizumab, steady state is achieved at and after ˜16 weeks of administration.

AUCss, Cavg,ss and Cmin,ss are pharmacokinetic measures of the systemic exposure to the drug (e.g. pembrolizumab) in humans after its administration, and are typically considered drivers of drug efficacy. AUCss and Cavg,ss represent the average exposure over a dosing interval, but differ in terms of units. “Cmin,ss” represents the minimum or lowest (trough) drug concentration observed at the end of a dosing interval, just before the next dose is administered.

“Cmax,ss” is the maximum or highest (peak) drug concentration observed soon after its administration. In the specific case of pembrolizumab, which is administered as intravenous infusion, the peak concentration occurs immediately after end of infusion. Cmax,ss is a metric that is typically considered a driver of safety.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Anti-tumor response” when referring to a cancer patient treated with a therapeutic regimen, such as a combination therapy described herein, means at least one positive therapeutic effect, such as for example, reduced number of cancer cells, reduced tumor size, reduced rate of cancer cell infiltration into peripheral organs, reduced rate of tumor metastasis or tumor growth, or progression free survival. Positive therapeutic effects in cancer can be measured in a number of ways (See, W. A. Weber, J. Null. Med. 50:1S-10S (2009); Eisenhauer et al., supra). In some embodiments, an anti-tumor response to a combination therapy described herein is assessed using RECIST 1.1 criteria, bidimensional irRC or unidimensional irRC. In some embodiments, an anti-tumor response is any of SD, PR, CR, PFS, or DFS. “Bidimensional irRC” refers to the set of criteria described in Wolchok J D, et al. Guidelines for the evaluation of immune therapy activity in solid tumors: immune-related response criteria. Clin Cancer Res. 2009; 15(23):7412-7420. These criteria utilize bidimensional tumor measurements of target lesions, which are obtained by multiplying the longest diameter and the longest perpendicular diameter (cm²) of each lesion. “Biotherapeutic agent” means a biological molecule, such as an antibody or fusion protein, that blocks ligand/receptor signaling in any biological pathway that supports tumor maintenance and/or growth or suppresses the anti-tumor immune response. Classes of biotherapeutic agents include, but are not limited to, antibodies to VEGF, EGFR, Her2/neu, other growth factor receptors, CD20, CD40, CD-40L, CTLA-4, OX-40, 4-1BB, and ICOS. “CBR” or “Clinical Benefit Rate” means CR+PR+durable SD.

“CDR” or “CDRs” as used herein means complementarity determining region(s) in an immunoglobulin variable region, defined using the Kabat numbering system, unless otherwise indicated.

“Chemotherapeutic agent” is a chemical compound useful in the treatment of cancer. Classes of chemotherapeutic agents include, but are not limited to alkylating agents, antimetabolites, kinase inhibitors, spindle poison plant alkaloids, cytoxic/antitumor antibiotics, topisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, and anti-sense oligonucleotides that inhibit expression of genes implicated in abnormal cell proliferation or tumor growth. Chemotherapeutic agents useful in the treatment methods of the present invention include cytostatic and/or cytotoxic agents. “Chothia” as used herein means an antibody numbering system described in Al-Lazikani et al., JMB 273:927-948 (1997).

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 1 below.

TABLE 1
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, a PD-1 antagonist that consists essentially of a recited amino acid sequence may also include one or more amino acids, including substitutions of one or more amino acid residues, which do not materially affect the properties of the binding compound.

“DCR” or “Disease Control Rate” means CR+PR+SD.

“Diagnostic anti-PD-L monoclonal antibody” means a mAb which specifically binds to the mature form of the designated PD-L (PD-L1 or PDL2) that is expressed on the surface of certain mammalian cells. A mature PD-L lacks the presecretory leader sequence, also referred to as leader peptide The terms “PD-L” and “mature PD-L” are used interchangeably herein, and shall be understood to mean the same molecule unless otherwise indicated or readily apparent from the context.

As used herein, a diagnostic anti-human PD-L1 mAb or an anti-hPD-L1 mAb refers to a monoclonal antibody that specifically binds to mature human PD-L1. A mature human PD-L1 molecule consists of amino acids 19-290 of the following sequence:

(SEQ ID NO: 32)
MRIFAVFIFMTYWHLLNAFTVTVPKDLYVVEYGSN
MTIECKFPVEKQLDLAALIVYWEMEDKNIIQFVHG
EEDLKVQHSSYRQRARLLKDQLSLGNAALQITDVK
LQDAGVYRCMISYGGADYKRITVKVNAPYNKINQR
ILVVDPVTSEHELTCQAEGYPKAEVIWTSSDHQVL
SGKTTTTNSKREEKLFNVTSTLRINTTTNEIFYCT
FRRLDPEENHTAELVIPELPLAHPPNERTHLVILG
AILLCLGVALTFIFRLRKGRMMDVKKCGIQDTNSK
KQSDTHLEET.

Specific examples of diagnostic anti-human PD-L1 mAbs useful as diagnostic mAbs for immunohistochemistry (IHC) detection of PD-L1 expression in formalin-fixed, paraffin-embedded (FFPE) tumor tissue sections are antibody 20C3 and antibody 22C3, which are described in WO2014/100079. Table 2 below provides characteristics of antibody 22C3. Another anti-human PD-L1 mAb that has been reported to be useful for IHC detection of PD-L1 expression in FFPE tissue sections (Chen, B. J. et al., Clin Cancer Res 19: 3462-3473 (2013)) is a rabbit anti-human PD-L1 mAb publicly available from Sino Biological, Inc. (Beijing, P.R. China; Catalog number 10084-R015).

TABLE 2
Characteristics of monoclonal
antibody MEB037.22C3 (22C3)

| | | |
|---|---|---|
| Antibody | | EQ ID |
| Feature | Amino Acid Sequence | NO |

Light Chain

| | | |
|---|---|---|
| CDRL1 | KSSQSLLHTSTRKNYLA | 13 |
| CDRL2 | WASTRES | 14 |
| CDRL3 | KQSYDVVT | 15 |
| Mature | DIVMSQSPSSLAVSAGE | 16 |
| Variable | KVTMTCKSSQSLLHTST | |
| Region | RKNYLAWYQQKPGQSPK | |
| | LLIYWASTRESGVPDRF | |
| | TGSGSGTDFTLTISSVQ | |
| | AEDLAVYYCKQSYDVVT | |
| | FGAGTKLELK | |

Heavy Chain

| | | |
|---|---|---|
| CDRH1 Kabat | SYWIH | 17 |
| Def'n | | |
| CDRH1 Chothia | GYTFTSYWIH | 18 |
| Def'n | | |
| CDRH2 | YINPSSGYHEYNQKFID | 19 |
| CDRH3 | SGWLIHGDYYFDF | 20 |
| Mature | XVHLQQSGAELAKPGAS | 21 |
| Variable | VKMSCKASGYTFTSYWI | |
| Region | HWIKQRPGQGLEWIGYI | |
| | NPSSGYHEYNQKFIDKA | |
| | TLTADRSSSTAYMHLTS | |
| | LTSEDSAVYYCARSGWL | |
| | IHGDYYFDFWGQGTTLT | |
| | VSS, | |
| | wherein X = Q or | |
| | pE (pyro-glutamate) | |

“PD-L1” or “PD-L2” expression as used herein means any detectable level of expression of the designated PD-L protein on the cell surface or of the designated PD-L mRNA within a cell or tissue. PD-L protein expression may be detected with a diagnostic PD-L antibody in an IHC assay of a tumor tissue section or by flow cytometry.

Alternatively, PD-L protein expression by 5 tumor cells may be detected by PET imaging, using a binding agent (e.g., antibody fragment, affibody and the like) that specifically binds to the desired PD-L target, e.g., PD-L1 or PD-L2. Techniques for detecting and measuring PD-L mRNA expression include RT-PCR, real-time quantitative RT-PCR, RNAseq, and the Nanostring platform (J. Clin. Invest. 2017; 127(8):2930-2940).

Several approaches have been described for quantifying PD-L1 protein expression in IHC assays of tumor tissue sections. See, e.g., Thompson, R. H., et al., PNAS 101 (49); 17174-17179 (2004); Thompson, R. H. et al., Cancer Res. 66:3381-3385 (2006); Gadiot, J., et al., Cancer 117:2192-2201 (2011); Taube, J. M. et al., Sci Transl Med 4, 127ra37 (2012); and Toplian, S. L. et al., New Eng. J Med. 366 (26): 2443-2454 (2012). See US 20170285037 which describes 15 Hematoxylin and Eosin staining used by the pathologist.

One approach employs a simple binary end-point of positive or negative for PD-L1 expression, with a positive result defined in terms of the percentage of tumor cells that exhibit histologic evidence of cell-surface membrane staining. A tumor tissue section is counted as positive for PD-L1 expression if it is at least 1% of total tumor cells.

In another approach, PD-L1 expression in the tumor tissue section is quantified in the tumor cells as well as in infiltrating immune cells, which predominantly comprise lymphocytes. The percentage of tumor cells and infiltrating immune cells that exhibit membrane staining are separately quantified as <5%, 5 to 9%, and then in 10% increments up to 100%. PD-L1 expression in the immune infiltrate is reported as a semi-quantitative measurement called the adjusted inflammation score (AIS), which is determined by multiplying the percent of membrane staining cells by the intensity of the infiltrate, which is graded as none (0), mild (score of 1, rare lymphocytes), moderate (score of 2, focal infiltration of tumor by lymphohistiocytic aggregates), or severe (score of 3, diffuse infiltration). A tumor tissue section is counted as positive for PD-L1 expression by immune infiltrates if the MS is ≥5.

The level of PD-L mRNA expression may be compared to the mRNA expression levels of one or more reference genes that are frequently used in quantitative RT-PCR.

In some embodiments, a level of PD-L1 expression (protein and/or mRNA) by malignant cells and/or by infiltrating immune cells within a tumor is determined to be “overexpressed” or elevated” based on comparison with the level of PD-L1 expression (protein and/or mRNA) by an appropriate control. For example, a control PD-L1 protein or mRNA expression level may be the level quantified in nonmalignant cells of the same type or in a section from a matched normal tissue. In some preferred embodiments, PD-L1 expression in a tumor sample is determined to be elevated if PD-L1 protein (and/or PD-L1 mRNA) in the sample is at least 10%, 20%, or 30% greater than in the control.

“Tumor proportion score (TPS)” refers to the percentage of tumor cells expressing PD-L1 on the cell membrane at any intensity (weak, moderate or strong). Linear partial or complete cell membrane staining is interpreted as positive for PD-L1.

“Mononuclear inflammatory density score (MIDS)” refers to the ratio of the number of PD-L1 expressing mononuclear inflammatory cells (MIC) infiltrating or adjacent to the tumor (small and large lymphocytes, monocytes, and macrophages within the tumor nests and the adjacent supporting stroma) compared to the total number of tumor cells. The MIDS is recorded at a scale from 0 to 4 with 0=none; 1=present, but less than one MIC for every 100 tumor cells (<1%); 2=at least one MIC for every 100 tumor cells, but less than one MIC per 10 tumor cells (1-9%); 3=at least one MIC for every 10 tumor cells, but fewer MIC's than tumor cells (10-99%); 4=at least as many MIC's as tumor cells (≥100%).

“Combined positive score (CPS)” refers to the ratio of the number of PD-L1 positive tumor cells and PD-L1 positive mononuclear inflammatory cells (MIC) within the tumor nests and the adjacent supporting stroma (numerator) compared to the total number of tumor cells (denominator; i.e., the number of PD-L1 positive and PD-L1 negative tumor cells). PD-L1 expression at any intensity is considered positive, i.e., weak (1+), moderate (2+), or strong (3+).

“PD-L1 expression positive” refers to a Tumor Proportion Score, Mononuclear Inflammatory Density Score or Combined Positive Score of at least 1%; AIS is >5; or elevated level of PD-L1 expression (protein and/or mRNA) by malignant cells and/or by infiltrating immune cells within a tumor compared to an appropriate control.

“DSDR” or “Durable Stable Disease Rate” means SD for >23 weeks. “Framework region” or “FR” as used herein means the immunoglobulin variable regions excluding the CDR regions.

“Kabat” as used herein means an immunoglobulin alignment and numbering system pioneered by Elvin A. Kabat ((1991) Sequences of Proteins of Immunological Interest, 5th Ed.

Public Health Service, National Institutes of Health, Bethesda, Md.).

“Anti-TIGIT antibody” means a monoclonal antibody that specifically binds to human TIGIT. Human TIGIT comprises the amino acid sequence:

(SEQ ID NO: 33)
MRWCLLLIWA QGLRQAPLAS GMMTGTIETT
GNISAEKGGS IILQCHLSST TAQVTQVNWE
QQDQLLAICN ADLGWHISPS FKDRVA
PGPGLGLTLQSLTV NDTGEYFCIY
HTYPDGTYTGRIFLEVLESS VAEHGARFQI
PLLGAMAATL VVICTAVIVV VALTRKKKAL
RIHSVEGDLRRKSAGQEEWS PSAPSPPGSC
VQAEAAPAGL CGEQRGEDCA ELHDYFNVLS
YRSLGNCSFF TETG;

See also amino acid residues 25-244 of Genbank Accession Number NP 776160.2 (SEQ ID NO: 33) (amino acid residues 1-24 of SEQ ID NO:33 correspond to a leader peptide).

“Microsatellite instability (MSI)” refers to the form of genomic instability associated with defective DNA mismatch repair in tumors. See Boland et al., Cancer Research 58, 5258-5257, 1998. In one embodiment, MSI analysis can be carried out using the five National Cancer Institute (NCI) recommended microsatellite markers BAT25 (GenBank accession no. 9834508), BAT26 (GenBank accession no. 9834505), D5S346 (GenBank accession no. 181171), D2S123 (GenBank accession no. 187953), D17S250 (GenBank accession no. 177030). Additional markers for example, BAT40, BAT34C4, TGF-β-RII and ACTC can be used. Commercially available kits for MSI analysis include, for example, the Promega MSI multiplex PCR assay.

“High frequency microsatellite instability” or “microsatellite instability-high (MSI-H)” refers to if two or more of the five NCI markers show instability or ≥30-40% of the total markers demonstrate instability (i.e. have insertion/deletion mutations).

“Low frequency microsatellite instability” or “microsatellite instability-low (MSI-L)” refers to if one of the five NCI markers show instability or <30-40% of the total markers exhibit instability (i.e. have insertion/deletion mutations).

“Non-MSI-H colorectal cancer” as used herein refers to microsatellite stable (MSS) and low frequency MSI (MSI-L) colorectal cancer.

“Microsatellite Stable (MSS)” refers to if none of the five NCI markers show instability (i.e. have insertion/deletion mutations)

“Proficient mismatch repair (pMMR) colorectal cancer” refers to normal expression of MMR proteins (MLH1, PMS2, MSH2, and MSH6) in a CRC tumor specimen by IHC. 10 Commercially available kits for MMR analysis include the Ventana MMR IHC assay.

“Mismatch repair deficient (dMMR) colorectal cancer” refers to low expression of one or more MMR protein(s) (MLH1, PMS2, MSH2, and MSH6) in a CRC tumor specimen by IHC.

“Monoclonal antibody” or “mAb” or “Mab”, as used herein, refers to a population of substantially homogeneous antibodies, i.e., the antibody molecules comprising the population are 15 identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a 20 substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

“Non-responder patient”, when referring to a specific anti-tumor response to treatment with a combination therapy described herein, means the patient did not exhibit the anti-tumor response.

“ORR” or “objective response rate” refers in some embodiments to CR+PR, and ORR_{(week 24)} refers to CR and PR measured using irRECIST in each patient in a cohort after 24 weeks of anti-cancer treatment. “Patient” or “subject” refers to any single subject for which therapy is desired or that is participating in a clinical trial, epidemiological study or used as a control, including humans and mammalian veterinary patients such as cattle, horses, dogs, and cats.

“PD-1 antagonist” means any chemical compound or biological molecule that blocks binding of PD-L1 expressed on a cancer cell to PD-1 expressed on an immune cell (T cell, B cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the treatment method, medicaments and uses of the present invention in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively.

As used herein, a “pembrolizumab variant” means a monoclonal antibody which comprises heavy chain and light chain sequences that are substantially identical to those in pembrolizumab, except for having three, two or one conservative amino acid substitutions at positions that are located outside of the light chain CDRs and six, five, four, three, two or one conservative amino acid substitutions that are located outside of the heavy chain CDRs, e.g., the variant positions are located in the FR regions or the constant region, and optionally has a deletion of the C-terminal lysine residue of the heavy chain. In other words, pembrolizumab and a pembrolizumab variant comprise identical CDR sequences, but differ from each other due to having a conservative amino acid substitution at no more than three or six other positions in their full length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

As used herein, the term “pemetrexed” refers to a compound named 5-substituted pyrrolo[2,3-d]pyrimidine. Specifically, the term refers to a multitargeted antifolate that exhibits anticancer effects against various cancers, including non-small cell lung cancer and malignant pleural mesothelioma. Pemetrexed exhibits anticancer effects against various cancers, including non-small cell lung cancer and malignant pleural mesothelioma, by inhibiting the activity of metabolites that are involved in folate metabolism. Pemetrexed analogs and variants may also be used. See PCT Publication number WO2014084651A1.

“RECIST 1.1 Response Criteria” as used herein means the definitions set forth in Eisenhauer et al., E. A. et al., Eur. J Cancer 45:228-247 (2009) for target lesions or nontarget lesions, as appropriate based on the context in which response is being measured.

“Responder patient” when referring to a specific anti-tumor response to treatment with a combination therapy described herein, means the patient exhibited the anti-tumor response.

“Sustained response” means a sustained therapeutic effect after cessation of treatment with a therapeutic agent, or a combination therapy described herein. In some embodiments, the sustained response has a duration that is at least the same as the treatment duration, or at least 1.5, 2.0, 2.5 or 3 times longer than the treatment duration.

Taxol is a valuable cancer chemotherapeutic agent used for treatment of many types of cancer, including ovary, breast, and lung carcinomas. Taxol is a natural product derived from the bark of Taxus brevafolio (Pacific yew). Taxol inhibits microtubule epolymerization during mitosis and results in subsequent cell death. Taxol displays a broad spectrum of tumoricidal activity including against breast, ovary and lung cancer (McGuire et al., 1996, N. Engld. J. Med. 334:1-6; and Johnson et al., 1996, J. Clin. Ocol. 14:2054-2060). While taxol is often effective in treatment of these malignancies, it is usually not curative because of eventual development of taxol resistance. Cellular resistance to taxol may include mechanisms such as enhanced expression of P-glycoprotein and alterations in tubulin structure through gene mutations in the beta chain or changes in the ratio of tubulin isomers within the polymerized microtubule (Wahl et al., 1996, Nature Medicine 2:72-79; Horwitz et al., 1993, Natl. Cancer Inst. 15:55-61; Haber et al., 1995, J. Biol. Chem. 270:31269-31275; and Giannakakou et al., 1997, J. Biol. Chem. 272:17118-17125). Some tumors acquires taxol resistance through unknown mechanisms.

“Tissue Section” refers to a single part or piece of a tissue sample, e.g., a thin slice of tissue cut from a sample of a normal tissue or of a tumor.

“Treat” or “treating” cancer as used herein means to administer therapeutic agents of the invention to a subject having cancer, or diagnosed with cancer, to achieve at least one positive therapeutic effect, such as for example, reduced number of cancer cells, reduced tumor size, reduced rate of cancer cell infiltration into peripheral organs, or reduced rate of tumor metastasis or tumor growth. Positive therapeutic effects in cancer can be measured in a number of ways (See, W. A. Weber, J. Nucl. Med. 50:1S-10S (2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In some embodiments, response to a combination therapy described herein is assessed using RECIST 1.1 criteria or irRC (bidimensional or unidimensional) and the treatment achieved by a combination of the invention is any of PR, CR, OR, PFS, DFS and OS. PFS, also referred to as “Time to Tumor Progression” indicates the length of time during and after treatment that the cancer does not grow, and includes the amount of time patients have experienced a CR or PR, as well as the amount of time patients have experienced SD. DFS refers to the length of time during and after treatment that the patient remains free of disease. OS refers to a prolongation in life expectancy as compared to naive or untreated individuals or patients. In some embodiments, response to a combination of the invention is any of PR, CR, PFS, DFS, OR and OS that is assessed using RECIST 1.1 response criteria. The treatment regimen for a combination of the invention that is effective to treat a cancer patient may vary according to factors such as the disease state, age, and weight of the patient, and the ability of the therapy to elicit an anti-cancer response in the subject.

While an embodiment of any of the aspects of the invention may not be effective in achieving a positive therapeutic effect in every subject, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

The terms “treatment regimen”, “dosing protocol” and “dosing regimen” are used interchangeably to refer to the dose and timing of administration of each therapeutic agent in a combination of the invention.

“Tumor” as it applies to a subject diagnosed with, or suspected of having, cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

“Tumor burden” also referred to as “tumor load”, refers to the total amount of tumor material distributed throughout the body. Tumor burden refers to the total number of cancer cells or the total size of tumor(s), throughout the body, including lymph nodes and bone marrow. Tumor burden can be determined by a variety of methods known in the art, such as, e.g. by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

“Unidimensional irRC refers to the set of criteria described in Nishino M, Giobbie-Hurder A, Gargano M, Suda M, Ramaiya N H, Hodi F S. Developing a Common Language for Tumor Response to Immunotherapy: Immune-related Response Criteria using Unidimensional measurements. Clin Cancer Res. 2013; 19(14):3936-3943). These criteria utilize the longest diameter (cm) of each lesion.

“Variable regions” or “V region” as used herein means the segment of IgG chains which is variable in sequence between different antibodies. It extends to Kabat residue 109 in the light chain and 113 in the heavy chain.

### Pd-1 Antagonists and Anti-Tigit Antibodies

PD-1 antagonists useful in the treatment method, medicaments and uses of the present invention include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)₂, scFv and Fv fragments. The anti-PD-1 or anti-PD-L1 antibody may be produced in CHO cells using conventional cell culture and recovery/purification technologies.

Examples of mAbs that bind to human PD-1, and useful in the treatment method, medicaments and uses of the present invention, are described in U.S. Pat. No. 7,488,802, U.S. Pat. Nos. 7,521,051, 8,008,449, 8,354,509, 8,168,757, WO2004/004771, WO2004/072286, WO2004/056875, and US2011/0271358. Specific anti-human PD-1 mAbs useful as the PD-1 antagonist in the treatment method, medicaments and uses of the present invention include: pembrolizumab (also known as MK-3475), a humanized IgG4 mAb with the structure described in WHO Drug Information, Vol. 27, No. 2, pages 161-162 (2013) and which comprises the heavy and light chain amino acid sequences shown in Table 3; nivolumab (BMS-936558), a human IgG4 mAb with the structure described in WHO Drug Information, Vol. 27, No. 1, pages 68-69 (2013) and which comprises the heavy and light chain amino acid sequences shown in Table 3; the humanized antibodies h409A11, h409A16 and h409A17, which are described in WO2008/156712, and AMP-514, which is being developed by MedImmune.

Examples of mAbs that bind to human PD-L1, and useful in the treatment method, medicaments and uses of the present invention, are described in PCT Publication numbers WO2013/019906 and WO2010/077634 A1 and U.S. Pat. No. 8,383,796. Specific anti-human PD-L1 mAbs useful as the PD-1 antagonist in the treatment method, medicaments and uses of the present invention include MPDL3280A, BMS-936559, MEDI4736, MSB0010718C and an antibody which comprises the heavy chain and light chain variable regions of SEQ ID NO:24 and SEQ ID NO:21, respectively, of WO2013/019906.

Other PD-1 antagonists useful in the treatment method, medicaments and uses of the present invention include an immunoadhesin that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in WO2010/027827 and WO2011/066342. Specific fusion proteins useful as the PD-1 antagonist in the treatment method, medicaments and uses of the present invention include AMP-224 (also known as B7-DCIg), which is a PD-L2-FC fusion protein and binds to human PD-1.

In some preferred embodiments of the treatment method, medicaments and uses of the present invention, the PD-1 antagonist is a monoclonal antibody, or antigen binding fragment thereof, which comprises: (a) light chain CDRs SEQ ID NOs: 1, 2 and 3 and (b) heavy chain CDRs SEQ ID NOs: 6, 7 and 8.

In other preferred embodiments of the treatment method, medicaments and uses of the present invention, the PD-1 antagonist is a monoclonal antibody, or antigen binding fragment thereof, which specifically binds to human PD-1 and comprises (a) a heavy chain variable region comprising SEQ ID NO:9 or a variant thereof, and (b) a light chain variable region comprising SEQ ID NO:4 or a variant thereof. A variant of a heavy chain variable region sequence is identical to the reference sequence except having up to 17 conservative amino acid substitutions in the framework region (i.e., outside of the CDRs), and preferably has less than ten, nine, eight, seven, six or five conservative amino acid substitutions in the framework region. A variant of a light chain variable region sequence is identical to the reference sequence except having up to five conservative amino acid substitutions in the framework region (i.e., outside of the CDRs), and preferably has less than four, three or two conservative amino acid substitution in the framework region.

In another preferred embodiment of the treatment method, medicaments and uses of the present invention, the PD-1 antagonist is a monoclonal antibody which specifically binds to human PD-1 and comprises (a) a heavy chain comprising SEQ ID NO: 10 and (b) a light chain comprising SEQ ID NO:5.

In yet another preferred embodiment of the treatment method, medicaments and uses of the present invention, the PD-1 antagonist is a monoclonal antibody which specifically binds to human PD-1 and comprises (a) a heavy chain comprising SEQ ID NO: 12 and (b) a light chain comprising SEQ ID NO:11.

In all of the above treatment method, medicaments and uses, the PD-1 antagonist inhibits the binding of PD-L1 to PD-1, and preferably also inhibits the binding of PD-L2 to PD-1. In some embodiments of the above treatment method, medicaments and uses, the PD-1 antagonist is a monoclonal antibody, or an antigen binding fragment thereof, which specifically binds to PD-1 or to PD-L1 and blocks the binding of PD-L1 to PD-1. In one embodiment, the PD-1 antagonist is an anti-PD-1 antibody which comprises a heavy chain and a light chain, and wherein the heavy and light chains comprise the amino acid sequences in SEQ ID NO:10 and SEQ ID NO:5, respectively.

Table 3 below provides a list of the amino acid sequences of exemplary anti-PD-1 mAbs for use in the treatment method, medicaments and uses of the present invention.

TABLE 3
Exemplary PD-1 Antibody Sequences

| | | |
|---|---|---|
| Antibody | Amino Acid | SEQ ID |
| Feature | Sequence | NO. |

Pembrolizumab Light Chain

| | | |
|---|---|---|
| CDR1 | RASKGVSTSGYSYLH | 1 |
| CDR2 | LASYLES | 2 |
| CDR3 | QHSRDLPLT | 3 |
| Variable | EIVLTQSPATLSLSPGE | 4 |
| Region | RATLSCRASKGVSTSGY | |
| | SYLHWYQQKPGQAPRLL | |
| | IYLASYLESGVPARFSG | |
| | SGSGTDFTLTISSLEPE | |
| | DFAVYYCQHSRDLPLTF | |
| | GGGTKVEIK | |
| Light Chain | EIVLTQSPATLSLSPGE | 5 |
| | RATLSCRASKGVSTSGY | |
| | SYLHWYQQKPGQAPRLL | |
| | IYLASYLESGVPARFSG | |
| | SGSGTDFTLTISSLEPE | |
| | DFAVYYCQHSRDLPLTF | |
| | GGGTKVEIKRTVAAPSV | |
| | FIFPPSDEQLKSGTASV | |
| | VCLLNNFYPREAKVQWK | |
| | VDNALQSGNSQESVTEQ | |
| | DSKDSTYSLSSTLTLSK | |
| | ADYEKHKVYACEVTHQG | |
| | LSSPVTKSFNRGEC | |

Pembrolizumab Heavy Chain

| | | |
|---|---|---|
| CDR1 | NYYMY | 6 |
| CDR2 | GINPSNGGTNFNEKFKN | 7 |
| CDR3 | RDYRFDMGFDY | 8 |
| Variable | QVQLVQSGVEVKKPGAS | 9 |
| Region | VKVSCKASGYTFTNYYM | |
| | YWVRQAPGQGLEWMGGI | |
| | NPSNGGTNFNEKFKNRV | |
| | TLTTDSSTTTAYMELKS | |
| | LQFDDTAVYYCARRDYR | |
| | FDMGFDYWGQGTTVTVS | |
| | S | |
| Heavy | QVQLVQSGVEVKKPGAS | 10 |
| Chain | VKVSCKASGYTFTNYYM | |
| | YWVRQAPGQGLEWMGGI | |
| | NPSNGGTNFNEKFKNRV | |
| | TLTTDSSTTTAYMELKS | |
| | LQFDDTAVYYCARRDYR | |
| | FDMGFDYWGQGTTVTVS | |
| | SASTKGPSVFPLAPCSR | |
| | STSESTAALGCLVKDYF | |
| | PEPVTVSWNSGALTSGV | |
| | HTFPAVLQSSGLYSLSS | |
| | VVTVPSSSLGTKTYTCN | |
| | VDHKPSNTKVDKRVESK | |
| | YGPPCPPCPAPEFLGGP | |
| | SVFLFPPKPKDTLMISR | |
| | TPEVTCVVVDVSQEDPE | |
| | VQFNWYVDGVEVHNAKT | |
| | KPREEQFNSTYRVVSVL | |
| | TVLHQDWLNGKEYKCKV | |
| | SNKGLPSSIEKTISKAK | |
| | GQPREPQVYT | |
| | LPPSQEEMTKNQVSLTC | |
| | LVKGFYPSDIAVEWESN | |
| | GQPENNYKTTPPVLDSD | |
| | GSFFLYSRLTVDKSRWQ | |
| | EGNVFSCSVMHEALHNH | |
| | YTQKSLSLSLGK | |

Nivolumab Light Chain

| | | |
|---|---|---|
| Light Chain | EIVLTQSPATLSLSPGE | 11 |
| | RATLSCRASQSVSSYLA | |
| | WYQQKPGQAPRLLIYDA | |
| | SNRATGIPARFSGSGSG | |
| | TDFTLTISSLEPEDFAV | |
| | YYCQQSSNWPRTFGQGT | |
| | KVEIKRTVAAPSVFIFP | |
| | PSDEQLKSGTASVVCLL | |
| | NNFYPREAKVQWKVDNA | |
| | LQSGNSOESVTEQDSKD | |
| | STYSLSSTLTLSKADYE | |
| | KHKVYACEVTHQGLSSP | |
| | VTKSFNRGEC | |

Nivolumab Heavy Chain

| | | |
|---|---|---|
| Heavy Chain | QVQLVESGGGVVQPGRS | 12 |
| | LRLDCKASGITFSNSGM | |
| | HWVRQAPGKGLEWVAVI | |
| | WYDGSKRYYADSVKGRF | |
| | TISRDNSKNTLFLQMNS | |
| | LRAEDTAVYYCATNDDY | |
| | WGQGTLVTVSSASTKGP | |
| | SVFPLAPCSRSTSESTA | |
| | ALGCLVKDYFPEPVTVS | |
| | WNSGALTSGVHTFPAVL | |
| | QSSGLYSLSSVVTVPSS | |
| | SLGTKTYTCNVDHKPSN | |
| | TKVDKRVESKYGPPCPP | |
| | CPAPEFLGGPSVFLFPP | |
| | KPKDTLMISRTPEVTCV | |
| | VVDVSQEDPEVQFNWYV | |
| | DGVEVHNAKTKPREEQF | |
| | NSTYRVVSVLTVLHODW | |
| | LNGKEYKCKVSNKGLPS | |
| | SIEKTISKAKGQPREPQ | |
| | VYTLPPSQEEMTKNQVS | |
| | LTCLVKGFYPSDIAVEW | |
| | ESNGQPENNYKTTPPVL | |
| | DSDGSFFLYSRLTVDKS | |
| | RWQEGNVFSCSVMHEAL | |
| | HNHYTQKSLSLSLGK | |

The anti-TIGIT antibody used in the claimed invention may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region.

In one embodiment, the anti-TIGIT antibody is 31C6. In another embodiment, the anti-TIGIT antibody is a 31C6 variant. The 31C6 antibody is a monoclonal antibody which contains two heavy chain and two light chains, wherein each heavy chain comprises the amino acid sequence of SEQ ID NO: 23 and each light chain comprises the amino acid sequence of SEQ ID NO: 22.

31C6: a light chain immunoglobulin comprising
the amino acid sequence:
(SEQ ID NO: 22)
DIQMTQSPSSLSASVGDRVTITCRASEHIYSYLSWYQQKPGKVPK
LLIYNAKTLAEGVPSRFSGSGSGTDFTLTISSLQPEDVATYYCQH
HFGSPLTFGQGTRLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC;
and
a heavy chain immunoglobulin comprising the
amino acid sequence:
(SEQ ID NO: 23)
EVQLVQSGAEVKKPGSSVKVSCKASGYTFSSYVMHWVRQAPGQG
LEWIGYIDPYNDGAYAQKFQGRVTLTSDKSTSTAYMELSSLRSE
DTAVYYCARGGPYGWYFDVWGQGTTVTVSSASTKGPSVFPLAPS
SKSTSGGTAALGCLVKDYFPEPVTVSWNSGALTSGVHTFPAVLQ
SSGLYSLSSVVTVPSSSLGTQTYICNVNHKPSNTKVDKKVEPKS
CDKTHTCPPCPAPELLGGPSVFLFPPKPKDTLMISRTPEVTCVV
VDVSHEDPEVKFNWYVDGVEVHNAKTKPREQYNSTYRVVSVLTV
LHQDWLNGKEYKCKVSNKALPAPIEKTISKAKGQPREPQVYTLP
PSRDELTKNQVSLTCLVKGFYPSDIAVEWESNGQPENNYKTTPP
VLDSDGSFFLYSKLTVDKSRWQQGNVFSCSVMHEALHNHYTQKS
LSLSPGK;
or
a light chain immunoglobulin variable domain
comprising the amino acid sequence:
(SEQ ID NO: 24 (CDRs underscored))
DIQMTQSPSSLSASVGDRVTITCRASEHIYSYLSWYQQKPGKVP
KLLIYNAKTLAEGVPSRFSGSGSGTDFTLTISSLQPEDVATYY
CQHHFGSPLTFGQGTRLEIK;
and
a heavy chain immunoglobulin variable domain
comprising the amino acid sequence:
(SEQ ID NO: 25 (CDRs underscored))
EVQLVQSGAEVKKPGSSVKVSCKASGYTFSSYVMHWVRQAPGQG
LEWIGYIDPYNDGAYAQKFQGRVTLTSDKSTSTAYMELSSLRSE
DTAVYYCARGGPYGWYFDVWGQGTTVTVSS;
or
comprising the CDRs:
CDR-L1:
(SEQ ID NO: 26)
RASEHIYSYLS;
CDR-L2:
(SEQ ID NO: 27)
NAKTLAE;
CDR-L3:
(SEQ ID NO: 28)
QHHFGSPLT;
CDR-H1:
(SEQ ID NO: 29)
SYVMH;
CDR-H2:
(SEQ ID NO: 30)
YIDPYNDGAKYAQKFQG;
and
CDR-H3:
(SEQ ID NO: 31)
GGPYGWYFDV

In some preferred embodiments of the treatment method, medicaments and uses of the present invention, the anti-TIGIT antibody comprises: (a) light chain CDRs of SEQ ID NOs: 26,30, 27 and 28 and (b) heavy chain CDRs of SEQ ID NOs: 29, 30 and 31.

In other preferred embodiments of the treatment method, medicaments and uses of the present invention, the anti-TIGIT antibody comprises (a) a heavy chain variable region comprising SEQ ID NO:25 or a variant thereof, and (b) a light chain variable region comprising SEQ ID NO:24 or a variant thereof. A variant of a heavy chain variable region sequence is identical to the reference sequence except having up to 17 conservative amino acid substitutions in the framework region (i.e., outside of the CDRs), and preferably has less than ten, nine, eight, seven, six or five conservative amino acid substitutions in the framework region. A variant of a light chain variable region sequence is identical to the reference sequence except having up to five conservative amino acid substitutions in the framework region (i.e., outside of the CDRs), and preferably has less than four, three or two conservative amino acid substitution in the framework region. Examples of such variants as set forth in international patent publication number WO2016/028656 (see, for example, SEQ ID NOs: 124-133 and 149-150 of international patent publication number WO2016/028656.

In another preferred embodiment of the treatment method, medicaments and uses of the present invention, the anti-TIGIT antibody comprises (a) a heavy chain comprising SEQ ID NO: 23 and (b) a light chain comprising SEQ ID NO:22. In another preferred embodiment of the treatment method, medicaments and uses of the present invention, the anti-TIGIT antibody comprises (a) a heavy chain variable region comprising SEQ ID NO: 25 and (b) a light chain variable region comprising SEQ ID NO:24.

In one embodiment, the anti-PD-1 or anti-TIGIT antibody or antigen-binding fragment comprises a heavy chain constant region, e.g. a human constant region, such as γ1, γ2, γ3, or γ4 human heavy chain constant region or a variant thereof. In another embodiment, the anti-PD-1 or anti-TIGIT antibody or antigen-binding fragment comprises a light chain constant region, e.g. a human light chain constant region, such as lambda or kappa human light chain region or variant thereof. By way of example, and not limitation, the human heavy chain constant region can be γ1 and the human light chain constant region can be kappa. In another embodiment, the human heavy chain constant region can be γ4 and the human light chain constant region can be kappa. In an alternative embodiment, the Fc region of the antibody is γ4 with a Ser228Pro mutation (Schuurman, J et. al., Mol. Immunol. 38: 1-8, 2001).

In some embodiments, different constant domains may be appended to humanized V_{L} and V_{H} regions derived from the CDRs provided herein. For example, if a particular intended use of an antibody (or fragment) of the present invention were to call for altered effector functions, a heavy chain constant domain other than human IgG1 may be used, or hybrid IgG1/IgG4 may be utilized. For example, a human IgG4 constant domain, for example, may be used. The present invention includes the use of anti-PD-1 antibodies or anti-TIGIT antibodies and antigen-binding fragments thereof which comprise an IgG4 constant domain. In one embodiment, the IgG4 constant domain can differ from the native human IgG4 constant domain (Swiss-Prot Accession No. P01861.1) at a position corresponding to position 228 in the EU system and position 241 in the KABAT system, where the native Ser108 is replaced with Pro, in order to prevent a potential inter-chain disulfide bond between Cys106 and Cys109 (corresponding to positions Cys 226 and Cys 229 in the EU system and positions Cys 239 and Cys 242 in the KABAT system) that could interfere with proper intra-chain disulfide bond formation. See Angal et al. (1993) Mol. Imunol. 30:105.

### Methods, Uses and Medicaments

In one aspect, the invention provides a method of treating cancer in a patient comprising administering an anti-TIGIT antibody at 2.1 mg to 700 mg, wherein the anti-TIGIT antibody comprises: (a) light chain CDRs of SEQ ID NOs: 26, 27 and 28 and (b) heavy chain CDRs of SEQ ID NOs: 29, 30 and 31. In one aspect, the anti-TIGIT antibody is administered via intravenous infusion. In another aspect, the invention provides a method of treating cancer in a patient comprising co-administering an anti-TIGIT antibody at 2.1 mg to 700 mg with an anti-PD-1 or anti-PD-L1 antibody, wherein the anti-TIGIT antibody comprises: (a) light chain CDRs of SEQ ID NOs: 26, 27 and 28 and (b) heavy chain CDRs of SEQ ID NOs: 29, 30 and 31. In one embodiment, the anti-PD-1 antibody blocks the binding of PD-1 to PD-L1 and PD-L2. In one embodiment, 2.1 mg to 700 mg of the anti-TIGIT antibody is administered. In various embodiments, 2.1 mg, 7 mg, 21 mg, 70 mg, 200 mg, 210 mg, or 700 mg of the anti-TIGIT antibody is administered. In another embodiment, 200-700 mg of the anti-TIGIT antibody is administered. In another embodiment, 200-700 mg of the anti-TIGIT antibody is administered. In another embodiment, 200 mg or 210 mg of the anti-TIGIT antibody is administered. In one aspect, the anti-TIGIT antibody is administered via intravenous infusion. In another aspect, the anti-PD-1 antibody or anti-PD-L1 antibody is administered via intravenous infusion. In a further embodiment, both the anti-TIGIT antibody and the anti-PD-1 antibody or anti-PD-L1 antibody are administered via intravenous infusion.

In a further aspect, the invention provides a method for treating cancer in a patient comprising administering via intravenous infusion to the individual a composition comprising 200 mg of pembrolizumab or a pembrolizumab variant and 200 mg of anti-TIGIT antibody 31C6 or a 31C6 variant. In another aspect, the invention provides a method for treating cancer in a patient comprising administering via intravenous infusion to the individual a composition comprising 200 mg of pembrolizumab or a pembrolizumab variant and 210 mg of anti-TIGIT antibody 31C6 or a 31C6 variant. In one embodiment, the composition comprises 200 mg of pembrolizumab or a pembrolizumab variant and 200-700 mg of anti-TIGIT antibody 31C6 or a 31C6 variant. In one embodiment, the composition comprises 200 mg of pembrolizumab or a pembrolizumab variant and 700 mg of anti-TIGIT antibody 31C6 or a 31C6 variant.

In another embodiment, the invention provides a medicament comprising the anti-TIGIT antibody for use in combination with an anti-PD-1 or anti-PD-L1 antibody for treating cancer, wherein the anti-TIGIT antibody is administered at 2.1 mg to 700 mg. In one embodiment, the anti-TIGIT antibody is administered via intravenous infusion. In another embodiment, the invention provides a medicament comprising the anti-TIGIT antibody and an anti-PD-1 antibody for treating cancer. In one embodiment, the medicament comprises 200 mg of pembrolizumab or a pembrolizumab variant and 200 mg of anti-TIGIT antibody 31C6 or a 31C6 variant. In another embodiment, the medicament comprises 200 mg of pembrolizumab or a pembrolizumab variant and 700 mg of 31C6 or a 31C6 variant.

In a still further embodiment, the invention provides use of the anti-TIGIT antibody and an anti-PD-1 or anti-PD-L1 antibody in the manufacture of a medicament for treating cancer in an individual. In one embodiment, the medicament comprises 200 mg of pembrolizumab or a pembrolizumab variant and 200 mg of anti-TIGIT antibody 31C6 or a 31C6 variant. In another aspect, the medicament comprises 200 mg of pembrolizumab or a pembrolizumab variant and 700 mg of 31C6 or a 31C6 variant. In a still further embodiment, the invention provides use of the anti-TIGIT antibody in the manufacture of a medicament for treating cancer in an individual, wherein the anti-TIGIT antibody is co-administered at 2.1 mg with the anti-PD-1 antibody at 200 mg. In one embodiment, each of the anti-TIGIT antibody and the anti-PD-1 antibody are administered via intravenous infusion.

In the foregoing methods, medicaments and uses, in one embodiment, the anti-PD-1 antibody and anti-TIGIT antibody are co-formulated. In one embodiment, a co-formulated product with 200 mg pembrolizumab or a pembrolizumab variant and 200 mg of antibody 31C6 or a 31C6 variant is used for intravenous infusion. In one embodiment, a co-formulated product with 200 mg pembrolizumab or a pembrolizumab variant and 300 mg of antibody 31C6 or a 31C6 variant is used for intravenous infusion. In one embodiment, a co-formulated product with 200 mg pembrolizumab or a pembrolizumab variant and 400 mg of antibody 31C6 or a 31C6 variant is used for intravenous infusion. In another embodiment, a co-formulated product with 200 mg of pembrolizumab or a pembrolizumab variant and 500 mg of antibody 31C6 or a 31C6 variant is used for intravenous infusion. In another embodiment, a co-formulated product with of 200 mg pembrolizumab or a pembrolizumab variant and 600 mg of antibody 31C6 or a 31C6 variant is used for intravenous infusion. In another embodiment, a co-formulated product with 200 mg of pembrolizumab or a pembrolizumab variant and 700 mg of antibody 31C6 or a 31C6 variant is used for intravenous infusion.

The invention also provides a pharmaceutical composition comprising 200 mg of pembrolizumab or a pembrolizumab variant, 200 mg of antibody 31C6 or a 31C6 variant, and one or more pharmaceutically acceptable excipients. In one embodiment, the pharmaceutical composition comprises 200 mg of pembrolizumab or a pembrolizumab variant, 300 mg of antibody 31C6 or a 31C6 variant, and one or more pharmaceutically acceptable excipients. In one embodiment, the pharmaceutical composition comprises 200 mg pembrolizumab or a pembrolizumab variant, 400 mg of antibody 31C6 or a 31C6 variant, and one or more pharmaceutically acceptable excipients. In another embodiment, the pharmaceutical composition comprises 200 mg of pembrolizumab or a pembrolizumab variant, 500 mg of antibody 31C6 or a 31C6 variant, and one or more pharmaceutically acceptable excipients. In a further embodiment, the pharmaceutical composition comprises 200 mg of pembrolizumab or a pembrolizumab variant, 600 mg of antibody 31C6 or a 31C6 variant, and one or more pharmaceutically acceptable excipients. In a further embodiment, the pharmaceutical composition comprises 200 mg of pembrolizumab or a pembrolizumab variant, 700 mg of antibody 31C6 or a 31C6variant, and one or more pharmaceutically acceptable excipients.

In the foregoing methods, medicaments and uses, in another embodiment, the anti-PD-1 or anti-PD-L1 antibody and anti-TIGIT antibody are co-administered. In one embodiment, 200 mg pembrolizumab or a pembrolizumab variant and 200 mg of antibody 31C6 or a 31C6 variant are co-administered on Day 1 and then every three weeks thereafter via intravenous infusion. In one embodiment, 200 mg of pembrolizumab or a pembrolizumab variant and 300 mg of antibody 31C6 or a 31C6 variant are co-administered on Day 1 and then once every three weeks thereafter via intravenous infusion. In one embodiment, 200 mg pembrolizumab or a pembrolizumab variant and 400 mg of antibody 31C6 or a 31C6 variant are co-administered on Day 1 and then once every three weeks thereafter via intravenous infusion. In another embodiment, 200 mg of pembrolizumab or a pembrolizumab variant and 500 mg 31C6 or a 31C6 variant are co-administered on Day 1 and then once every three weeks thereafter via intravenous infusion. In another embodiment, 200 mg of pembrolizumab or a pembrolizumab variant and 600 mg of antibody 31C6 or a 31C6 variant are co-administered on Day 1 and then once every three weeks thereafter via intravenous infusion. In a further embodiment, 200 mg of pembrolizumab or a pembrolizumab variant and 700 mg of antibody 31C6 or a 31C6 variant are co-administered on Day 1 and then once every three weeks thereafter via intravenous infusion.

In the foregoing methods, medicaments and uses, in one embodiment, 400 mg pembrolizumab or a pembrolizumab variant is administered on Day 1 and then every six weeks thereafter and 200 mg of antibody 31C6 or a 31C6 variant is administered on Day 1 and then once every weeks thereafter, each via intravenous infusion. In one embodiment, 400 mg of pembrolizumab or a pembrolizumab variant is administered on Day 1 and then once every six weeks thereafter and 300 mg of antibody 31C6 or a 31C6 variant is administered on Day 1 and then once every three weeks thereafter, each via intravenous infusion. In one embodiment, 400 mg of pembrolizumab or a pembrolizumab variant is administered on Day 1 and then once every six weeks thereafter and 400 mg of antibody 31C6 or a 31C6 variant is administered on Day 1 and then once every three weeks thereafter, each via intravenous infusion. In another embodiment, 400 mg of pembrolizumab or a pembrolizumab variant is administered on Day 1 and then once every six weeks thereafter and 500 mg of antibody 31C6 or a 31C6 variant is administered on Day 1 and then once every three weeks thereafter, each via intravenous infusion. In another embodiment, 400 mg of pembrolizumab or a pembrolizumab variant is administered on Day 1 and then once every six weeks thereafter and 600 mg of antibody 31C6 or a 31C6 variant is administered on Day 1 and then once every three weeks thereafter, each via intravenous infusion. In another embodiment, 400 mg of pembrolizumab or a pembrolizumab variant is administered on Day 1 and then once every six weeks thereafter and 700 mg of antibody 31C6 or a 31C6 variant is administered on Day 1 and then once every three weeks thereafter, each via intravenous infusion.

Cancers that may be treated by the antibodies, compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal:

esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma) colorectal; Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; Hematologic: blood (myeloid leukemia [acute and chronic], acute lymphoblastic leukemia, chronic lymphocytic leukemia, myeloproliferative diseases, multiple myeloma, myelodysplastic syndrome); hematopoietic tumors of the lymphoid lineage, include leukemia, acute lymphocytic leukemia, chronic lymphocytic leukemia, acute lymphoblastic leukemia, B-cell lymphoma, T-cell lymphoma, Hodgkins lymphoma, non-Hodgkins lymphoma, hairy cell lymphoma, mantle cell lymphoma, myeloma, and Burkett's lymphoma; hematopoetic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma, and schwannomas; and other tumors, including melanoma, skin (non-melanomal) cancer, mesothelioma (cells), seminoma, teratocarcinoma, osteosarcoma, xenoderoma pigmentosum, keratoctanthoma, thyroid follicular cancer and Kaposi's sarcoma. In one embodiment, the forgoing cancers are advanced, unresectable or metastatic. In one embodiment, the patients are refractory to anti-PD-1 or anti-PD-L1 therapy.

In one embodiment, cancers that may be treated by the antibodies, compositions and methods of the invention include, but are not limited to: lung cancer, pancreatic cancer, colon cancer, colorectal cancer, myeloid leukemias, acute myelogenous leukemia, chronic myelogenous leukemia, chronic myelomonocytic leukemia, thyroid cancer, myelodysplastic syndrome, bladder carcinoma, epidermal carcinoma, melanoma, breast cancer, prostate cancer, head and neck cancers, ovarian cancer, brain cancers, cancers of mesenchymal origin, sarcomas, tetracarcinomas, neuroblastomas, kidney carcinomas, hepatomas, non-Hodgkin's lymphoma, multiple myeloma, and anaplastic thyroid carcinoma.

In another embodiment, cancers that may be treated by the antibodies, compositions and methods of the invention include, but are not limited to: head and neck squamous cell cancer, gastric cancer, adenocarcinoma of the stomach and/or gastric-esophageal junction, renal cell cancer, fallopian tube cancer, endometrial cancer, cervical cancer, and colorectal cancer. In one embodiment, the colorectal cancer, gastric cancer, adenocarcinoma of the stomach and/or gastric-esophageal junction (GEJ), or endometrial cancer is non-microsatellite instability-high (non-MSI-H) or proficient mismatch repair (pMMR). In one embodiment, the patient with head and neck squamous cell cancer is anti-PD-1 or anti-PD-L1 treatment refractory. In one embodiment, the colorectal cancer is unresectable or metastatic (Stage IV).

In another embodiment, cancers that may be treated by the antibodies, compositions and methods of the invention include hematological malignancies, but are not limited to: classical Hodgkin lymphoma (cHL), diffuse large B-cell lymphoma (DLBCL), transformed DLBCL, gray zone lymphoma, double hit lymphoma, Primary mediastinal B cell lymphoma (PMBCL) or indolent non-Hodgkin lymphoma (iNHL) (for example, follicular lymphoma, marginal zone lymphoma, mucosa-associated lymphoid tissue lymphoma, or small lymphocytic lymphoma). In one embodiment, the patient with Hodgkin lymphoma is anti-D-1 or anti-PD-L1 treatment refractory.

In a further embodiment, cancers that may be treated by the antibodies, compositions and methods of the invention include cancers selected from the group consisting of: renal cell carcinoma, urothelial carcinoma of the renal pelvis, ureter, bladder or urethra, melanoma, gastric, GEJ adenocarcinoma non-small cell lung cancer and bladder cancer. In one embodiment, the forgoing cancers are advanced, unresectable or metastatic.

In one embodiment, the non-small cell lung cancer is advanced or Stage IV. In another embodiment, the melanoma is advanced or Stage III. In one embodiment, the patients are refractory to anti-PD-1 or anti-PD-L1 therapy.

In one embodiment, a co-formulated product with pembrolizumab or a pembrolizumab variant and 31C6 or a 31C6 variant is used. In a further embodiment, a co-formulated product with 200 mg pembrolizumab or a pembrolizumab variant and 200 mg 31C6 or a 31C6 variant is used. In another embodiment, a co-formulated product with 200 mg pembrolizumab or a pembrolizumab variant and 700 mg 31C6 or a 31C6 variant is used.

In a further embodiment, the cancer is non-small cell lung cancer, and the patient lacks tumor activating epidermal growth factor receptor (EGFR), or B isoform of rapidly accelerated fibrosarcoma (B-Raf) mutations and lacks anaplastic lymphoma kinase (ALK) or c-ros oncogene 1 (ROS1) gene rearrangements. In a further embodiment, the cancer is non-small cell lung cancer, and the tumor has a squamous histology.

The combination therapy may also comprise one or more additional therapeutic agents. The additional therapeutic agent may be, e.g., a chemotherapeutic, a biotherapeutic agent, an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), and cells transfected with genes encoding immune stimulating cytokines such as but not limited to GM-CSF). The specific dosage and dosage schedule of the additional therapeutic agent can further vary, and the optimal dose, dosing schedule and route of administration will be determined based upon the specific therapeutic agent that is being used.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gamma1I and calicheamicin phiI1, see, e.g., Agnew, Chem. Intl. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above.

Each therapeutic agent in a combination therapy of the invention may be administered either alone or in a medicament (also referred to herein as a pharmaceutical composition) which comprises the therapeutic agent and one or more pharmaceutically acceptable carriers, excipients and diluents, according to standard pharmaceutical practice.

Each therapeutic agent in a combination therapy of the invention may be administered simultaneously (i.e., in the same medicament), concurrently (i.e., in separate medicaments administered one right after the other in any order) or sequentially in any order. Sequential administration is particularly useful when the therapeutic agents in the combination therapy are in different dosage forms (one agent is a tablet or capsule and another agent is a sterile liquid) and/or are administered on different dosing schedules, e.g., a chemotherapeutic that is administered at least daily and a biotherapeutic that is administered less frequently, such as once weekly, once every two weeks, or once every three weeks.

In some embodiments, the anti-TIGIT antibody is administered before administration of the anti-PD-1 antibody or anti-PD-L1 antibody, while in other embodiments, the anti-TIGIT antibody is administered after administration of the anti-PD-1 antibody or anti-PD-L1 antibody. In another embodiment, the anti-TIGIT antibody is administered concurrently with the anti-PD-15 antibody or anti-PD-L1 antibody.

In some embodiments, at least one of the therapeutic agents in the combination therapy is administered using the same dosage regimen (dose, frequency and duration of treatment) that is typically employed when the agent is used as monotherapy for treating the same cancer. In other embodiments, the patient receives a lower total amount of at least one of the therapeutic agents in the combination therapy than when the agent is used as monotherapy, e.g., smaller doses, less frequent doses, and/or shorter treatment duration.

Each small molecule therapeutic agent in a combination therapy of the invention can be administered orally or parenterally, including the intravenous, intramuscular, intraperitoneal, subcutaneous, rectal, topical, and transdermal routes of administration. A combination therapy of the invention may be used prior to or following surgery to remove a tumor and may be used prior to, during or after radiation therapy.

In some embodiments, a combination therapy of the invention is administered to a patient who has not been previously treated with a biotherapeutic or chemotherapeutic agent, i.e., is treatment-naïve. In other embodiments, the combination therapy is administered to a patient who failed to achieve a sustained response after prior therapy with a biotherapeutic or chemotherapeutic agent, i.e., is treatment-experienced.

A combination therapy of the invention is typically used to treat a tumor that is large enough to be found by palpation or by imaging techniques well known in the art, such as MRI, ultrasound, or CAT scan. A combination therapy of the invention can be administered to a human patient who has a cancer that tests positive for one or both of PD-L1 and PD-L2, and preferably tests positive for PD-L1 expression. In some preferred embodiments, PD-L1 expression is detected using a diagnostic anti-human PD-L1 antibody, or antigen binding fragment thereof, in an IHC assay on an FFPE or frozen tissue section of a tumor sample removed from the patient. Typically, the patient's physician would order a diagnostic test to determine PD-L1 expression in a tumor tissue sample removed from the patient prior to initiation of treatment with the anti-PD-1 antibody or anti-PD-L1 antibody and anti-TIGIT antibody, but it is envisioned that the physician could order the first or subsequent diagnostic tests at any time after initiation of treatment, such as for example after completion of a treatment cycle. In one embodiment, the PD-L1 expression is measured by the PD-L1 IHC 22C3 pharmDx assay. In another embodiment, the patient has a Mononuclear Inflammatory Density Score for PD-L1 expression≥2. In another embodiment, the patient has a Mononuclear Inflammatory Density Score for PD-L1 expression≥3. In another embodiment, the patient has a Mononuclear Inflammatory Density Score for PD-L1 expression≥4. In another embodiment, the patient has a Tumor Proportion Score for PD-L1 expression≥1%. In another embodiment, the patient has a Tumor Proportion Score for PD-L1 expression 20≥10%. In another embodiment, the patient has a Tumor Proportion Score for PD-L1 expression≥20%. In another embodiment, the patient has a Tumor Proportion Score for PD-L1 expression≥30%. In a further embodiment, the patient has a Combined Positive Score for PD-L1 expression≥1%. In a further embodiment, the patient has a Combined Positive Score for PD-L1 expression between 1 and 20%. In a further embodiment, the patient has a Combined Positive Score for PD-L1 expression≥2%. In a further embodiment, the patient has a Combined Positive Score for PD-L1 expression≥5%. In yet a further embodiment, the patient has a Combined Positive Score for PD-L1 expression≥10%. In a further embodiment, the patient has a Combined Positive Score for PD-L1 expression≥15%. In yet a further embodiment, the patient has a Combined Positive Score for PD-L1 expression≥20%.

In one preferred embodiment of the invention, the anti-PD-1 antibody in the combination therapy is nivolumab, which is administered intravenously at a dose selected from the group consisting of: 1 mg/kg Q2W, 2 mg/kg Q2W, 3 mg/kg Q2W, 5 mg/kg Q2W, 10 mg Q2W, 1 mg/kg Q3W, 2 mg/kg Q3W, 3 mg/kg Q3W, 5 mg/kg Q3W, and 10 mg/kg Q3W.

In another preferred embodiment of the invention, the anti-PD-1 antibody in the combination therapy is pembrolizumab, or a pembrolizumab variant, which is administered in a liquid medicament at a dose selected from the group consisting of 1 mg/kg Q2W, 2 mg/kg Q2W, 3 mg/kg Q2W, 5 mg/kg Q2W, 10 mg/kg Q2W, 1 mg/kg Q3W, 2 mg/kg Q3W, 3 mg/kg Q3W, 5 mg/kg Q3W, 10 mg/kg Q3W and flat-dose equivalents of any of these doses, i.e., such as 200 mg Q3W. In some embodiments, pembrolizumab is provided as a liquid medicament which comprises 25 mg/ml pembrolizumab, 7% (w/v) sucrose, 0.02% (w/v) polysorbate 80 in 10 mM histidine buffer pH 5.5. In other embodiments, pembrolizumab is provided as a liquid medicament which comprises about 125 to about 200 mg/mL of pembrolizumab, or antigen binding fragment thereof; about 10 mM histidine buffer; about 10 mM L-methionine, or a pharmaceutically acceptable salt thereof; about 7% (w/v) sucrose; and about 0.02% (w/v) polysorbate 80.

In some embodiments of the invention, the anti-PD-1 antibody, or antigen binding fragment thereof, is administered to the patient once every four or six weeks for 12 weeks or more. In other embodiments, the anti-PD-1 antibody, or antigen binding fragment thereof, is administered to the patient once every six weeks for 16 weeks or more, 18 weeks or more, 20 weeks or more, 24 weeks or more, 28 weeks or more, 30 weeks or more, 32 weeks or more, 36 weeks or more, 40 weeks or more, 42 weeks or more, 44 weeks or more, 48 weeks or more, 52 weeks or more, 54 weeks or more, 56 weeks or more, 60 weeks or more, 64 weeks or more, 66 weeks or more, 68 weeks or more, 72 weeks or more, 76 weeks or more, 78 weeks or more, 80 weeks or more, 84 weeks or more, 88 weeks or more, or 90 weeks or more. In other embodiments, the anti-PD-1 antibody, or antigen binding fragment thereof, is administered at 400 mg every six weeks.

In some embodiments, the selected dose of pembrolizumab is administered by IV infusion. In one embodiment, the selected dose of pembrolizumab is administered by IV infusion over a time period of between 25 and 40 minutes, or about 30 minutes.

In some embodiments, the patient is treated with the combination therapy for at least 24 weeks, e.g., eight 3-week cycles. In some embodiments, treatment with the combination therapy continues until the patient exhibits evidence of PD or a CR.

Pharmaceutically acceptable excipients of the present disclosure include for instance, solvents, bulking agents, buffering agents, tonicity adjusting agents, and preservatives (see, e.g., Pramanick et al., Pharma Times, 45:65-77, 2013). In some embodiments the pharmaceutical compositions may comprise an excipient that functions as one or more of a solvent, a bulking agent, a buffering agent, and a tonicity adjusting agent (e.g., sodium chloride in saline may serve as both an aqueous vehicle and a tonicity adjusting agent). The pharmaceutical compositions of the present disclosure are suitable for parenteral administration.

In some embodiments, the pharmaceutical compositions comprise an aqueous vehicle as a solvent. Suitable vehicles include for instance sterile water, saline solution, phosphate buffered saline, and Ringer's solution. In some embodiments, the composition is isotonic.

The pharmaceutical compositions may comprise a bulking agent. Bulking agents are particularly useful when the pharmaceutical composition is to be lyophilized before administration. In some embodiments, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The pharmaceutical compositions may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the composition within a range of 4 to 9. In some embodiments, the pH is greater than (lower limit) 4, 5, 6, 7 or 8. In some embodiments, the pH is less than (upper limit) 9, 8, 7, 6 or 5. That is, the pH is in the range of from about 4 to 9 in which the lower limit is less than the upper limit.

The pharmaceutical compositions may comprise a tonicity adjusting agent. Suitable tonicity adjusting agents include for instance dextrose, glycerol, sodium chloride, glycerin and mannitol.

The pharmaceutical compositions may comprise a preservative. Suitable preservatives include for instance antioxidants and antimicrobial agents. However, in preferred embodiments, the pharmaceutical composition is prepared under sterile conditions and is in a single use container, and thus does not necessitate inclusion of a preservative.

In some embodiments, a medicament comprising an anti-PD-1 antibody as the PD-1 antagonist may be provided as a liquid formulation or prepared by reconstituting a lyophilized powder with sterile water for injection prior to use. WO 2012/135408 describes the preparation of liquid and lyophilized medicaments comprising pembrolizumab that are suitable for use in the present invention. In some embodiments, a medicament comprising pembrolizumab is provided in a glass vial which contains about 100 mg of pembrolizumab in 4 ml of solution. Each 1 mL of solution contains 25 mg of pembrolizumab and is formulated in: L-histidine (1.55 mg), polysorbate 80 (0.2 mg), sucrose (70 mg), and Water for Injection, USP. The solution requires dilution for IV infusion.

In some embodiments, a medicament comprising the anti-TIGIT antibody may be provided as a liquid formulation or prepared by reconstituting a lyophilized powder with sterile water for injection prior to use. In one embodiment, the liquid formulation comprises about 10-100 mg/mL anti-TIGIT antibody; about 7% (w/v) sucrose; about 0.02% (w/v) polysorbate 80; about 10 mM L-histidine buffer at about pH 5.8-6.0; and about 10 mM to about 15 mM L-methionine.

The medicaments described herein may be provided as a kit which comprises a first container and a second container and a package insert. The first container contains at least one dose of a medicament comprising a PD-1 antagonist, the second container contains 2.1-700 mg of a medicament comprising the anti-TIGIT antibody, and the package insert, or label, which comprises instructions for treating a patient for cancer using the medicaments. The first and second containers may be comprised of the same or different shape (e.g., vials, syringes and bottles) and/or material (e.g., plastic or glass). The kit may further comprise other materials that may be useful in administering the medicaments, such as diluents, filters, IV bags and lines, needles and syringes. In some preferred embodiments of the kit, the PD-1 antagonist is an anti-PD-1 antibody and the instructions state that the medicaments are intended for use in treating a patient having cancer that tests positive for PD-L1 expression by an IHC assay.
These and other aspects of the invention, including the exemplary specific embodiments listed below, will be apparent from the teachings contained herein.

### EXEMPLARY SPECIFIC EMBODIMENTS OF THE INVENTION

1. A method for treating cancer in a patient comprising administering to the patient 2.1 mg to 700 mg of an anti-TIGIT antibody comprising a heavy chain and a light chain, wherein the light chain comprises light chain CDRs of SEQ ID NOs: 26, 27 and 28 and the heavy chain comprises heavy chain CDRs of SEQ ID NOs: 29, 30 and 31.
2. The method of embodiment 1, wherein the anti-TIGIT antibody is administered via intravenous infusion.
3. The method of embodiment 1, wherein the patient is administered 2.1 mg of the anti-TIGIT antibody.
4. The method of embodiment 1, wherein the patient is administered 7 mg of the anti-TIGIT antibody.
5. The method of embodiment 1, wherein the patient is administered 21 mg of the anti-TIGIT antibody.
6. The method of embodiment 1, wherein the patient is administered 70 mg of the anti-TIGIT antibody.
7. The method of embodiment 1, wherein the patient is administered 200 mg of the anti-TIGIT antibody.
8. The method of embodiment 1, wherein the patient is administered 210 mg of the anti-TIGIT antibody.
9. The method of embodiment 1, wherein the patient is administered 700 mg of the anti-TIGIT antibody.
10. The method of any one of embodiments 1 to 9, wherein the patient is administered the anti-TIGIT antibody on Day 1 and then once every three weeks thereafter.
11. The method of any one of embodiments 1 to 10, wherein the heavy chain comprises a heavy chain variable region comprising SEQ ID NO:25 and the light chain comprises a light chain variable region comprising SEQ ID NO: 24.
12. The method of any one of embodiments 1 to 11, wherein the anti-TIGIT antibody comprises a heavy chain and a light chain, and wherein the heavy chain comprises SEQ ID NO:23 and the light chain comprises SEQ ID NO:22.
13. The method of any one of embodiments 1 to 10, wherein the anti-TIGIT antibody is a 31C6 variant.
14. The method of any one of embodiments 1 to 13, wherein the anti-TIGIT antibody is co-administered with an anti-PD-1 antibody or anti-PD-L1 antibody, or an antigen binding fragment thereof.
15. The method of any one of embodiments 1 to 13, wherein the anti-TIGIT antibody is co-formulated with an anti-PD-1 antibody or anti-PD-L1 antibody, or an antigen binding fragment thereof.
16. The method of embodiments 14 or 15, wherein the anti-PD-1 antibody, or antigen binding fragment thereof, specifically binds to human PD-1 and blocks the binding of human PD-L1 to human PD-1.
17. The method of embodiment 16, wherein the anti-PD-1 antibody, or antigen binding fragment thereof, also blocks binding of human PD-L2 to human PD-1.
18. The method of embodiment 17, wherein the anti-PD-1 antibody, or antigen binding fragment thereof, comprises: (a) light chain CDRs of SEQ ID NOs: 1, 2 and 3 and (b) heavy chain CDRs of SEQ ID NOs: 6, 7 and 8.
19. The method of embodiment 18, wherein the anti-PD-1 antibody comprises a heavy chain and a light chain, and wherein the heavy chain comprises a heavy chain variable region comprising SEQ ID NO:9 and the light chain comprises a light chain variable region comprising SEQ ID NO: 4.
20. The method of embodiment 19, wherein the anti-PD-1 antibody comprises a heavy chain and a light chain, and wherein the heavy chain comprises SEQ ID NO:10 and the light chain comprises SEQ ID NO:5.
21. The method of embodiment 17, wherein the anti-PD-1 antibody is pembrolizumab.
22. The method of embodiment 17, wherein the anti-PD-1 antibody is a pembrolizumab variant.
23. The method of embodiment 14, wherein the anti-PD-1 antibody is nivolumab.
24. The method of embodiment 14, wherein the anti-PD-L1 antibody is atezolizumab, durvalumab, or avelumab.
25. The method of any one of embodiments 18-24, wherein the anti-PD-1 antibody is administered at 200 mg via intravenous infusion on Day 1 and then once every three weeks thereafter.
26. The method of any one of embodiments 18-24, wherein the anti-PD-1 antibody is administered at 400 mg via intravenous infusion on Day 1 and then once every six weeks thereafter.
27. The method of embodiments 14 or 15 wherein the anti-PD-1 antibody is a humanized anti-PD-1 antibody that comprises a heavy chain and a light chain, and wherein the heavy chain comprises a heavy chain variable region comprising heavy chain CDRs of SEQ ID NOs: 6, 7 and 8 and the light chain comprises a light chain variable region comprising light chain CDRs of SEQ ID NOs: 1, 2 and 3; and the anti-TIGIT antibody is a humanized anti-TIGIT antibody which comprises a heavy chain and a light chain, and wherein the heavy chain comprises a heavy chain variable region comprising heavy chain CDRs of SEQ ID NOs: 29, 30 and 31 and the light chain comprises a light chain variable region comprising light chain CDRs of SEQ ID NOs: 26, 27 and 28.
28. The method of embodiments 14 or 15 wherein the anti-PD-1 antibody comprises a heavy chain and a light chain, and wherein the heavy chain comprises a heavy chain variable region comprising SEQ ID NO:9 and the light chain comprises a light chain variable region comprising SEQ ID NO: 4; and the anti-TIGIT antibody comprises a heavy chain and a light chain, and wherein the heavy chain comprises a heavy chain variable region comprising SEQ ID NO:25 and the light chain comprises a light chain variable region comprising SEQ ID NO: 24.
29. The method of embodiments 14 or 15 wherein the anti-PD-1 antibody comprises a heavy chain and a light chain, and wherein the heavy chain comprises SEQ ID NO:10 and the light chain comprises SEQ ID NO: 5; and the anti-TIGIT antibody comprises a heavy chain and a light chain, and wherein the heavy chain comprises SEQ ID NO:23 and the light chain comprises a light chain variable region comprising SEQ ID NO: 22.
30. The method of any one of embodiments 27-29, wherein the anti-PD-1 antibody is administered at 200 mg via intravenous infusion on Day 1 and then once every three weeks thereafter, and the anti-TIGIT antibody is administered at 200 mg via intravenous infusion on Day 1 and then once every three weeks thereafter.
31. The method of any one of embodiments 27-29, wherein the anti-PD-1 antibody is administered at 400 mg via intravenous infusion on Day 1 and then once every six weeks thereafter, and the anti-TIGIT antibody is administered at 200 mg via intravenous infusion on Day 1 once every three weeks.
32. The method of any one of embodiments 27-29, wherein 200 mg of anti-PD-1 antibody is co-formulated with 200 mg anti-TIGIT antibody.
33. The method of any one of embodiments 1 to 32, wherein the cancer is selected from the group consisting of: NSCLC, colorectal cancer, gastric cancer, breast cancer, cervical cancer, ovarian, epithelial, fallopian tube, or primary peritoneal carcinoma.
34. The method of embodiment 33, wherein the cancer is NSCLC.
35. The method of embodiment 34, the method further comprising administering a combination of (i) carboplatin and pemetrexed or (ii) carboplatin and paclitaxel.
36. The method of any one of embodiments 1 to 35, wherein the individual has not been previously treated with anti-PD-1 or anti-PD-L1 therapy or is confirmed progressive while receiving prior anti-PD-1 or anti-PD-L1 therapy.
37. A pharmaceutical composition comprising 200 mg pembrolizumab or a pembrolizumab variant, 200 mg of 31C6 or a 31C6 variant, and a pharmaceutically acceptable excipient.
38. Use of an anti-TIGIT antibody comprising a heavy chain and a light chain, wherein the light chain comprises light chain CDRs of SEQ ID NOs: 26, 27 and 28 and the heavy chain comprises heavy chain CDRs of SEQ ID NOs: 29, 30 and 31 in the manufacture of a medicament for treating cancer in a subject/patient.
39. The use of embodiment 38, wherein the anti-TIGIT antibody is formulated for and/or administered by intravenous infusion.
40. The use of embodiment 38 or 39, wherein the patient is administered 2.1 mg of the anti-TIGIT antibody.
41. The use of embodiment 38 or 39, wherein the patient is administered 7 mg of the anti-TIGIT antibody.
42. The use of embodiment 38 or 39, wherein the patient is administered 21 mg of the anti-TIGIT antibody.
43. The use of embodiment 38 or 39, wherein the patient is administered 70 mg of the anti-TIGIT antibody.
44. The use of embodiment 38 or 39, wherein the patient is administered 200 mg of the anti-TIGIT antibody.
45. The use of embodiment 38 or 39, wherein the patient is administered 210 mg of the anti-TIGIT antibody.
46. The use of embodiment 38 or 39, wherein the patient is administered 700 mg of the anti-TIGIT antibody.
47. The use of any one of embodiments 38 to 46 wherein the patient is administered the anti-TIGIT antibody on Day 1 and then once every three weeks thereafter.
48. The use of any one of embodiments 1 to 10, wherein the heavy chain comprises a heavy chain variable region comprising SEQ ID NO:25 and the light chain comprises a light chain variable region comprising SEQ ID NO: 24.
49. The use of any one of embodiments 38 to 48, wherein the anti-TIGIT antibody comprises a heavy chain and a light chain, and wherein the heavy chain comprises SEQID NO:23 and the light chain comprises SEQ ID NO:22.
50. The use of any one of embodiments 38 to 47, wherein the anti-TIGIT antibody is a 31C6 variant.
51. The use of any one of embodiments 38 to 50, wherein the anti-TIGIT antibody is co-administered with an anti-PD-1 antibody or anti-PD-L1 antibody, or an antigen binding fragment thereof.
52. The use of any one of embodiments 38 to 50, wherein the anti-TIGIT antibody is co-formulated with an anti-PD-1 antibody or anti-PD-L1 antibody, or an antigen binding fragment thereof.
53. The use of embodiments 51 or 52, wherein the anti-PD-1 antibody, or antigen binding fragment thereof, specifically binds to human PD-1 and blocks the binding of human PD-L1 to human PD-1.
54. The use of embodiment 53, wherein the anti-PD-1 antibody, or antigen binding fragment thereof, also blocks binding of human PD-L2 to human PD-1.
55. The use of embodiment 54, wherein the anti-PD-1 antibody, or antigen binding fragment thereof, comprises: (a) light chain CDRs of SEQ ID NOs: 1, 2 and 3 and (b) heavy chain CDRs of SEQ ID NOs: 6, 7 and 8.
56. The use of embodiment 55, wherein the anti-PD-1 antibody comprises a heavy chain and a light chain, and wherein the heavy chain comprises a heavy chain variable region comprising SEQ ID NO:9 and the light chain comprises a light chain variable region comprising SEQ ID NO: 4.
57. The use of embodiment 56, wherein the anti-PD-1 antibody comprises a heavy chain and a light chain, and wherein the heavy chain comprises SEQ ID NO:10 and the light chain comprises SEQ ID NO:5.
58. The use of any of embodiments 51 to 54, wherein the anti-PD-1 antibody is pembrolizumab.
59. The use of any of embodiments 51 to 54, wherein the anti-PD-1 antibody is a pembrolizumab variant.
60. The use of embodiment 51 or 52, wherein the anti-PD-1 antibody is nivolumab.
61. The method of embodiment 14, wherein the anti-PD-L1 antibody is atezolizumab, durvalumab, or avelumab.
62. The use of any one of embodiments 55-58, wherein the anti-PD-1 antibody is administered at 200 mg via intravenous infusion on Day 1 and then once every three weeks thereafter.
63. The use of any one of embodiments 55-58, wherein the anti-PD-1 antibody is administered at 400 mg via intravenous infusion on Day 1 and then once every six weeks thereafter.
64. The use of embodiments 51 or 52 wherein the anti-PD-1 antibody is a humanized anti-PD-1 antibody that comprises a heavy chain and a light chain, and wherein the heavy chain comprises a heavy chain variable region comprising heavy chain CDRs of SEQ ID NOs: 6, 7 and 8 and the light chain comprises a light chain variable region comprising light chain CDRs of SEQ ID NOs: 1, 2 and 3; and the anti-TIGIT antibody is a humanized anti-TIGIT antibody which comprises a heavy chain and a light chain, and wherein the heavy chain comprises a heavy chain variable region comprising heavy chain CDRs of SEQ ID NOs: 29, 30 and 31 and the light chain comprises a light chain variable region comprising light chain CDRs of SEQ ID NOs: 26, 27 and 28.
65. The use of embodiments 51 or 52 wherein the anti-PD-1 antibody comprises a heavy chain and a light chain, and wherein the heavy chain comprises a heavy chain variable region comprising SEQ ID NO:9 and the light chain comprises a light chain variable region comprising SEQ ID NO: 4; and the anti-TIGIT antibody comprises a heavy chain and a light chain, and wherein the heavy chain comprises a heavy chain variable region comprising SEQ ID NO:25 and the light chain comprises a light chain variable region comprising SEQ ID NO: 24.
66. The use of embodiments 51 or 52 wherein the anti-PD-1 antibody comprises a heavy chain and a light chain, and wherein the heavy chain comprises

SEQ ID NO:10 and the light chain comprises SEQ ID NO: 5; and the anti-TIGIT antibody comprises a heavy chain and a light chain, and wherein the heavy chain comprises SEQ ID NO:23 and the light chain comprises a light chain variable region comprising SEQ ID NO: 22.
67. The use of any one of embodiments 64 to 66, wherein the anti-PD-1 antibody is administered at 200 mg via intravenous infusion on Day 1 and then once every three weeks thereafter, and the anti-TIGIT antibody is administered at 200 mg via intravenous infusion on Day 1 and then once every three weeks thereafter.
68. The use of any one of embodiments 64 to 66, wherein the anti-PD-1 antibody is administered at 400 mg via intravenous infusion on Day 1 and then once every six weeks thereafter, and the anti-TIGIT antibody is administered at 200 mg via intravenous infusion on Day 1 once every three weeks.
69. The use of any one of embodiments 64 to 66, wherein 200 mg of anti-PD-1 antibody is co-formulated with 200 mg anti-TIGIT antibody.
70. The use of any one of embodiments 38 to 69, wherein the cancer is selected from the group consisting of: NSCLC, colorectal cancer, gastric cancer, breast cancer, cervical cancer, ovarian cancer, epithelial cancer, fallopian tube cancer, or primary peritoneal carcinoma.
71. The use of embodiment 70, wherein the cancer is NSCLC.
72. The use of any of embodiments 38 to 71 further comprising use of a combination of (i) carboplatin and pemetrexed or (ii) carboplatin and paclitaxel.
73. The use of any one of embodiments 38 to 72, wherein the individual has not been previously treated with anti-PD-1 or anti-PD-L1 therapy or is confirmed progressive while receiving prior anti-PD-1 or anti-PD-L1 therapy.

### General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protcols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York). Monoclonal, polyclonal, and humanized antibodies can be prepared (see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000)J. Immunol. 165:6205; He, et al. (1998) 1 Immunol. 160:1029; Tang et al. (1999) J. Biol. Chem. 274:27371-27378; Baca et al. (1997) 1 Biol. Chem. 272:10678-10684; Chothia et al. (1989) Nature 342:877-883; Foote and Winter (1992) J Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, N.Y.; Kay et al. (1996) Phage Display of Peptides and Proteins: A Laboratory Manual, Academic Press, San Diego, Calif.; de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fuse with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al., (1998) J. Immunol. 160:3891-3898; Hsing and Bishop (1999) J. Immunol. 162:2804-2811; Everts et al. (2002) J. Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J.). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, Oreg.; Sigma-Aldrich (2003) Catalogue, St. Louis, Mo.).

Standard methods of histology of the immune system are described (see, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

### EXAMPLES

### Example 1: Clinical Studies of Anti-TIGIT Antibody in Advanced Solid Tumors

This study was a multisite, open-label, dose-escalation study of anti-TIGIT antibody 31C6 monotherapy (Part A, Arm 1) and 31C6 in combination with pembrolizumab (Part A, Arm 2) Part B of the study is a dose confirmation phase to estimate the recommended Phase 2 dose for the 31C6 antibody when used as monotherapy and in combination with pembrolizumab. The anti-tumor activity of the 31C6 antibody when used as monotherapy and in combination with pembrolizumab in participants with advanced solid tumors was evaluated in Part B in a non-randomized study design. Part B evaluated 2 doses of 31C6 antibody in combination in participants with programmed death 1 (PD-1) treatment naive cancer using a 1:1 randomized study design.

During Part A of the study, subjects were allocated into 1 of 2 treatment arms:

Arm 1: 3106 as monotherapy escalating doses 2.1 mg, 7 mg, 21 mg, 70 mg, 210 mg, and 700 mg every 3 weeks (Q3W) via intravenous infusion (IV).

Arm 2: 3106 escalating doses 2.1 mg, 7 mg, 21 mg, 70 mg, 210 mg, and 700 mg every 3 weeks (Q3W) IV in combination with pembrolizumab (200 mg Q3W) IV.

Part A included adults with a metastatic solid tumor for which there is no clinically effective treatment who had measurable disease per RECIST and ECOG PS 0-1; previous CTLA-4, PD-1, or PD-L1 inhibitor treatment was permitted if it was not discontinued for an AE. Dose escalation followed a modified toxicity probability interval design with a target dose-limiting toxicity rate of −30%. Pembrolizumab was dosed at 200 mg Q3W. The anti-TIGIT 31C6 antibody was dosed at 2.1 mg to 700 mg. The anti-TIGIT antibody and pembrolizumab were given for 35 cycles or until progression, intolerable toxicity, or investigator or patient decision. During dose escalation, a minimum of 3 patients were required at each dose. Dose escalation and confirmation were complete after 14 patients were treated at any dose level. Primary end points are the safety a tolerability of the anti-TIGIT antibody 31C6 as monotherapy and in combination with pembrolizumab to establish the respective recommended phase 2 doses (RP2Ds). Secondary end points are the PK of the anti-TIGIT antibody 31C6 as monotherapy and in combination with pembrolizumab, the PK of pembrolizumab given with the anti-TIGIT antibody 31C6, and ORR (RECIST v1.1, investigator review) for the anti-TIGIT antibody 31C6 as monotherapy and with pembrolizumab.

Part B is a dose confirmation of 31C6 in combination with pembrolizumab. Additionally, expansion cohorts assessed the antitumor efficacy of 31C6 as monotherapy and in combination with pembrolizumab. Enrollment into the expansion portion of the study was open for subjects with the following cancers:
PD-1/PD-L1 inhibitor treatment-refractory NSCLC,
PD-1/PD-L1 inhibitor naive NSCLC,
colorectal cancer
breast cancer
ovarian epithelial, fallopian tube, or primary peritoneal carcinoma.

The Baseline characteristics for Part A of the study are set forth in the following tables: Baseline Characteristics: Table 4 (Data Cutoff date: Aug. 16, 2018)

TABLE 4
Baseline characteristics (Data Cutoff date: Aug. 16, 2018)

| Characteristic, | 31C6 monotherapy | | 31C6 + Pembrolizumab | |
|---|---|---|---|---|
| n (%) | N = 34 | | N = 34 | |
| Age, median (range) | 67.5 | (33-82) | 62.5 | (24-79) |
| Male sex | 16 | (47%) | 22 | (65%) |
| ECOG PS 1 | 17 | (50%) | 22 | (65%) |
| Prior Therapy | | | | |
| Neoadjuvant | 1 | (3%) | 0 | |
| Adjuvant | 2 | (6%) | 3 | (9%) |
| 1 | 3 | (9%) | 2 | (6%) |
| 2 | 9 | (26%) | 15 | (44%) |
| 3 | 5 | (15%) | 5 | (15%) |
| 4 | 8 | (24%) | 3 | (9%) |
| ≥5 | 5 | (15%) | 4 | (12%) |
| Missing | 1 | (3%) | 2 | (6%) |

TABLE 5
Baseline Characteristics (Data Cutoff Date: Aug. 16, 2018)

| Primary Cancer, | 31C6 monotherapy | 31C6 + Pembrolizumab |
|---|---|---|
| n (%) | N = 34 | N = 34 |
| NSCLC | 7 (21%) | 7 (21%) |
| Colorectal | 6 (18%) | 4 (12%) |
| Ovarian | 4 (12%) | 2 (6%) |
| Gastric/GEJ | 3 (9%) | 5 (15%) |
| Head and Neck | 3 (9%) | 0 |
| Thymic | 2 (6%) | 1 (3%) |
| Pancreatic | 1 (3%) | 2 (6%) |
| Urothelial | 1 (3%) | 2 (6%) |
| Breast | 0 | 2 (6%) |
| Sarcoma | 0 | 2 (6%) |
| Other | 5 (15%)^{a} | 5 (15%)^{b} |
| Missing/unknown | 2 (6%) | 2 (6%) |

^{a}Includes 1 patient each with esophageal, gallbladder, intestinal, mesothelioma and SCLC
^{b}Includes 1 patient each with melanoma, Merkel cell, RCC, squamous, and uterine.

### Results:

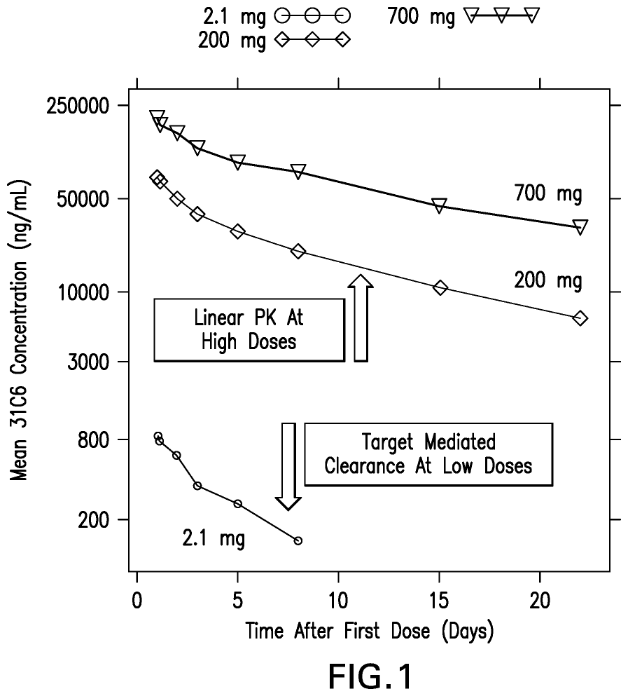

In this first-in-human study, the 31C6 anti-TIGIT antibody given as monotherapy and in combination with pembrolizumab (200 mg) was well tolerated and had manageable safety across all doses tested. Dose finding proceeded to completion without DLTs and there were no treatment related deaths. FIG. 1 sets forth the PK data from both the monotherapy and the combination therapy arms. The disposition of the patients was as follows for the monotherapy: 2 on treatment and 32 discontinued (27 with progressive disease, 2 on physician decision and 3 withdrawals). Thirteen (13) patients crossed over to combination therapy with pembrolizumab.

For the anti-TIGIT 31C6 and Pembrolizumab combination, dose escalation was completed for each prespecified dose level. No DLTs were observed. The disposition of the patients on combination therapy was as follows: 7 on treatment, 27 discontinued treatment (25 with progressive diseases, 1 physician decision and 1 withdrawal). A summary of the adverse events is listed below:

TABLE 6
Adverse Event Summary

| Adverse Event, | 31C6 monotherapy | 31C6 + Pembrolizumab |
|---|---|---|
| n (%) | N = 34 | N = 47^{a} |
| Any attribution | | |
| Any Grade | 33 (97%) | 45 (96%) |
| Grade 3-5 | 13 (38%) | 20 (43%) |
| Grade 5 | 1 (3%) | 3 (6%) |
| Led to Discontinuation | 0 | 1 (2%) |
| Treatment Related | | |
| Any Grade | 19 (56%) | 28 (60%) |
| Grade 3-5 | 2 (6%) | 5 (11%) |
| Grade 5 | 0 | 0 |
| Led to Discontinuation | 0 | 0 |

^{a}Includes the 34 patients originally allocated to the combination and the 13 who crossed over from 31C6 monotherapy.

TABLE 7
Treatment Related Adverse Events:

| 31C6 monotherapy | | 31C6 + Pembrolizumab | |
|---|---|---|---|
| N = 34 | | N = 47^{a} | |
| Occurred in ≥2 patients, n (%) | | Occurred in ≥2 patients, n (%) | |
| Fatigue | 5 (15%) | Pruritus | 10 (21%) |
| Pruritus | 4 (12%) | Fatigue | 4 (9%) |
| Anemia | 3 (9%) | Nausea | 4 (9%) |
| Infusion-related Reaction | 3 (9%) | Rash | 4 (9%) |
| Arthralgia | 2 (6%) | Decreased appetite | 3 (6%) |
| Decreased appetite | 2 (6%) | Diarrhea | 3 (6%) |
| Dermatitis acneiform | 2 (6%) | ALT increased | 2 (4%) |
| Diarrhea | 2 (6%) | Dyspnea | 2 (4%) |
| Headache | 2 (6%) | Hypophosphatemia | 2 (4%) |
| Nausea | 2 (6%) | Neuropathy peripheral | 2 (4%) |
| Rash | 2 (6%) | Pyrexia | 2 (4%) |
| Rash maculopapular | 2 (6%) | Rash maculopapular | 2 (4%) |

| | |
|---|---|
| 2 grade 3: anemia and diarrhea (n = 1 each) | 5 grade 3: ALT increased, colitis, γGT |
| 0 grade 4 | increased, hypersensitivity, and rash |
| | maculopapular (n = 1 each) |
| | 0 grade 4 |

^{a}Includes the 34 patients originally allocated to the combination and the 13 who crossed over from 31C6 monotherapy.
Data cutoff date: Aug. 16, 2018

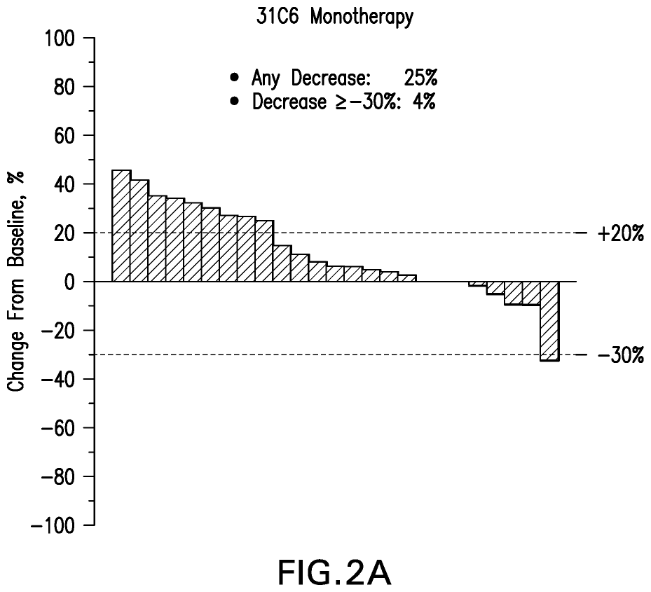

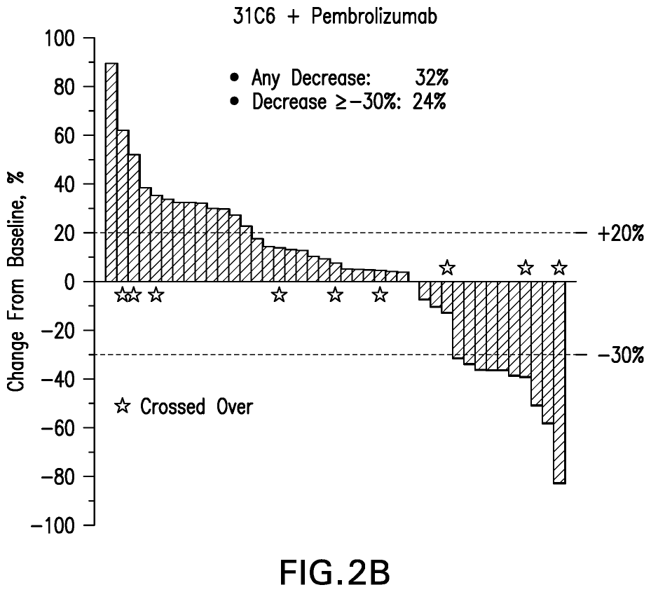

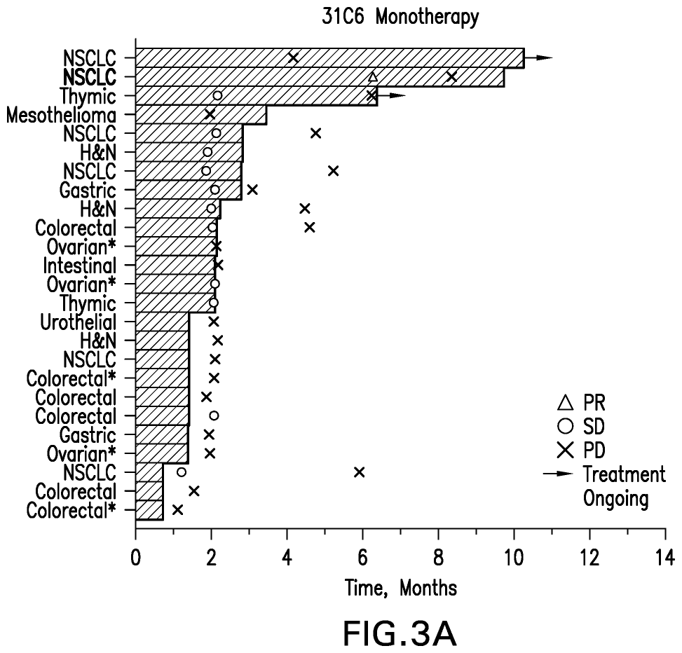

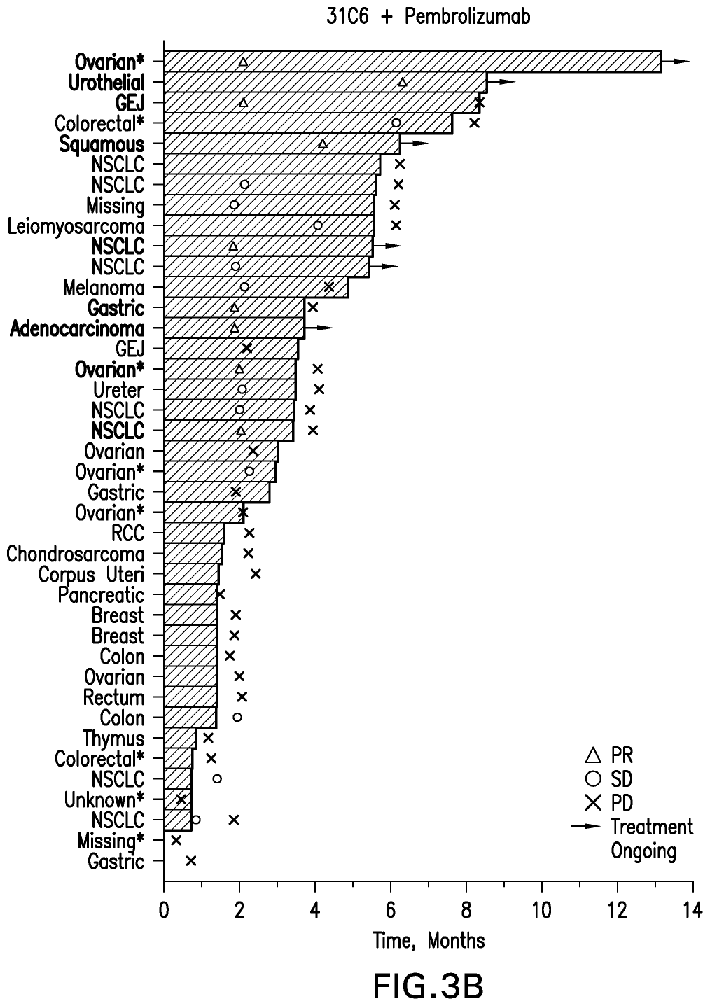

In addition, promising anti-tumor activity was observed in a heavily pretreated population across multiple tumor types, particular for combination therapy. Specifically, 3% ORR and 35% DCR was observed for 31C6 monotherapy alone and 19% ORR and 47% DCR was observed for the combination therapy of 31C6 and pembrolizumab. Responses were observed in patients who crossed over from monotherapy to combination therapy. FIG. 2A and FIG. 2B set forth the best percentage change from baseline in target lesions (RECIST v1.1, Investigator Review). FIG. 3A and FIG. 3B set forth a diagram of the treatment duration and response (RECIST v1.1, Investigator Review). A summary of the anti-tumor activity (RECIST v1.1, Investigator Review) is set forth below:

TABLE 8
Antitumor activity
Antitumor Activity^{a}

| | 31C6 monotherapy | | 31C6 + Pembrolizumab | |
|---|---|---|---|---|
| Response | N = 34 | | N = 43^{b} | |
| ORR, % (95% CI) | 3% | (<1-15) | 19% | (8-33) |
| DCR, % (95% CI) | 35% | (20-54) | 47% | (31-62) |
| Best response, n (%) | | | | |
| Complete Response | 0 | | 0 | |
| Partial Response | 1 | (3%) | 8 | (19%) |
| Stable disease | 11 | (32%) | 12 | (28%) |
| Progressive disease | 13 | (38%) | 20 | (47%) |
| Not assessed^{c} | 9 | (26%) | 3 | (7%) |

^{a}Evaluated in patients with measurable disease at baseline. Includes confirmed and unconfirmed responses.
^{b}includes the 34 patients originally allocated to the combination and the 13 who crossed over from 31C6 monotherapy.
^{c}no post-baseline assessment as of cutoff date. Data cut off date: Aug. 16, 2018

A partial response was observed in a 75 year old female patient with BRCA wild-type Ovarian cancer. The patient received 4 prior lines of chemotherapy and had no prior anti-PD-1 or anti-PD-L1 therapy. The patient received 31C6 anti-TIGIT antibody 2.1 mg monotherapy with document PD per RECIST, then crossed over to combination therapy of 2.1 mg of 31C6 anti-TIGIT antibody plus 200 mg of pembrolizumab. A partial response was observed 9 weeks after cross over. Specifically, there was a 85% reduction in tumor volume, reduction in size of all lesions (mesenteric deposits, lymph nodes (para-aortic, iliac, cervical)). Response was ongoing at 13 months. Treatment was discontinued because of rash.

### Example 2

### Part B of the Phase I Study

The design of Part B of the Phase I study described supra in example 1 is as follows:

TABLE 9
Design of Part B of the Phase 1 study

| Tumor Type | 31C6 Dose | Prior Therapy | Treatment Arm |
|---|---|---|---|
| NSCLC | 200 mg | PD-1 naïve | 31C6 |
| | | | Monotherapy |
| | | | 31C6 + |
| | | | Pembrolizumab |
| | | | (200 mg) |
| | | PD-1 refractory | 31C6 + |
| | | | Pembrolizumab |
| | | | (200 mg) |
| Breast | 200 mg | PD-1 naïve | 31C6 + |
| (at least 10 TNBC) | | | Pembrolizumab |
| | | | (200 mg) |
| Ovarian | 200 mg | PD-1 naïve | 31C6 + |
| (platinum resistant) | | | Pembrolizumab |
| | | | (200 mg) |
| CRC | 200 MG | PD-1 naïve | 31C6 + |
| | | | Pembrolizumab |
| | | | (200 mg) |
| Cervical | 200 MG | PD-1 naïve | 31C6 + |
| | | | Pembrolizumab |
| | | | (200 mg) |
| Cervical | 700 MG | PD-1 naive | 31C6 + |
| | | | Pembrolizumab |
| | | | (200 mg) |

In addition, the following combinations are being pursued:
31C6 + Pembrolizumab + Carboplatin and Paclitaxel (Taxol) in squamous PD1 Naïve
NSCLC 31C6 + Pembrolizumab + Carboplatin in nonsquamous PD1 Naïve NSCLC
31C6 + Pembrolizumab + Carboplatin and Taxol in squamous PD1 Naïve NSCLC

### Results:

### Overview of Safety

As of September 2019, it was observed that the 31C6 and pembrolizumab side effect profile was comparable to pembrolizumab alone. Adverse effect rates were similar between 31C6 monotherapy and the 31C6 and pembrolizumab combination therapy treatments. Adrenal insufficiency and infusion reactions occurred at higher rate than pembrolizumab Reflex sympathetic dystrophy (RSD). See overview Table 10.

TABLE 10
Overview of Adrenal insufficiency and infusion reactions

| | 31C6 | 31C6 + pembro | Pembrolizumab RSD |
|---|---|---|---|
| | (n = 68) | (n = 236) | (n = 2799) |
| Adrenal | 1, 1.5% (0.0, 7.9%) | 6, 2.5% (0.9, 5.5%) | 22, 0.8% (0.5, 1.2%) |
| insufficiency | | | |
| Infusion | 3, 4.4% (0.9, 12.4%) | 14, 5.9% (3.3, 9.8%) | 70, 2.5% (2.0, 3.1%) |
| reaction | | | |

It was observed that patients administered doses such as 200 mg had a linear clearance value consistent with previous observations in Part A data (200 mg: CL=0.49 L/day and t1/2=9.9 days; 700 mg: CL=0.48 L/day and t1/2=9.4 days). PK variability was also analyzed and showed consistent results (CV in CL=46%)

### Efficacy

Anti-tumor activity was analyzed in the different patient groups tested in Part B. PD-1 Naïve NSCLC

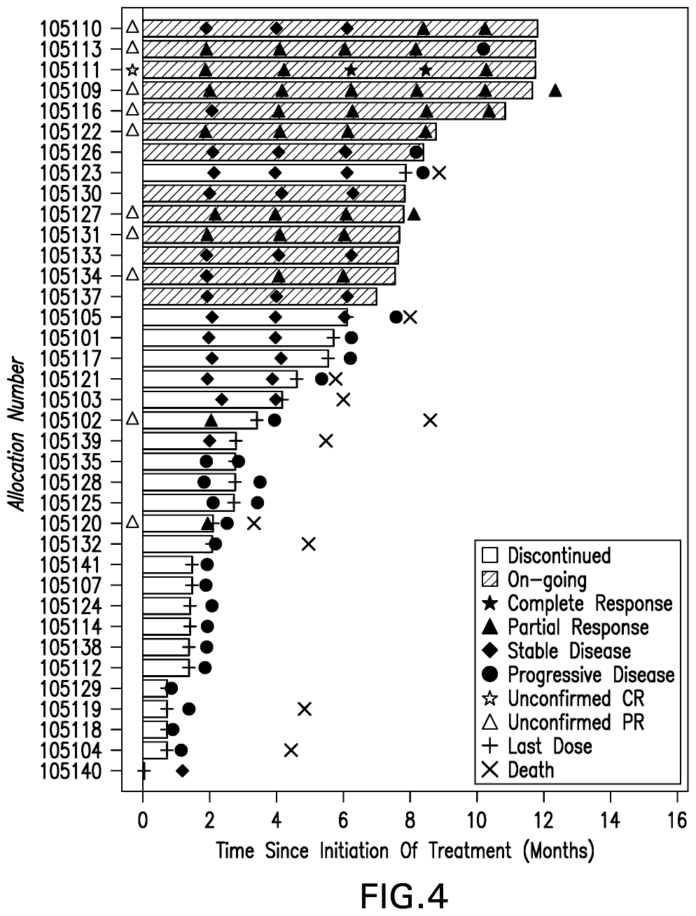

Responses were observed in naive NSCLC patients and are shown in FIG. 4 and Table 11 (RECIST v1.1, Investigator Review). The ORR data show that the combination of 31C6 and pembrolizumab is higher than control, despite enrolling patients with all PD-L1 levels and more advanced treatment line. In NSCLC subjects enrolled in KN-010 that had received one or more prior lines of therapy and were TPS≥1%, and ORR was 18%.

TABLE 11
Best Overall Response by RECIST1.1 by Investigator
in PD-1 Naïve NSCLC

| | Without | With |
|---|---|---|
| | confirmation | confirmation |
| Subjects in | 41 | 41 |
| population | | |
| CR | 1 (2%) | 1 (2%) |
| PR | 10 (24%) | 8 (20%) |
| ORR | 11 (27%) | 9 (22%) |
| SD | 12 (29%) | 13 (32%) |
| DCR | 23 (56%) | 22 (54%) |
| PD | 14 (34%) | 14 (34%) |
| NE or NA | 4 (10%) | 5 (12%) |

KN-010: 100% pts TPS ≥1, 29% pts are 3L+
Pembro + 31C6: 52% pts TPS ≥1, 60% subjects are 3L+

In addition, data was obtained showing the best response by RECIST1.1 by Investigator in subjects with PD-1 refractory NSCLC treated with either 31C6 antibody (200 mg) monotherapy or a combination therapy of 31C6 (200 mg) and pembrolizumab (200 m). See Table 12. Monotherapy activity was observed in unselected PD1 refractory NSCLC, also observed was a noticeable ORR result. Monotherapy responses were noted including a CR. Monotherapy results of ORR for 31C6 antibody (200 mg) were similar to the results for the combination therapy.

TABLE 12
Best Overall Response by RECIST1.1 by Investigator
in subjects with PD-1 refractory NSCLC treated with
31C6 alone or in combination with pembrolizumab

| Treatment | No. | | Without | With |
|---|---|---|---|---|
| Arm | Subjects | Response | confirmation | confirmation |
| 31C6 | 41 | | | |
| | | CR* | 1 (2%) | 1 (2%) |
| | | PR | 2 (5%) | 1 (2%) |
| | | ORR | 3 (7%) | 2 (5%) |
| | | SD | 13 (32%) | 13 (32%) |
| | | DCR | 16 (37%) | 15 (37%) |
| | | PD | 19 (46%) | 19 (46%) |
| | | NE or NA | 6 (15%) | 7 (15%) |
| 31C6 + Pembro | 37 | | | |
| | | CR | 0 (0%) | 0 (0%) |
| | | PR | 1 (3%)* | 1 (3%) |
| | | ORR | 1 (3%) | 1 (3%) |
| | | SD | 17 (46%) | 17 (46%) |
| | | DCR | 18 (49%) | 18 (49%) |
| | | PD | 16 (43%) | 16 (43%) |
| | | NE or NA | 3 (8%) | 3 (8%) |

83% of PD-1 refractory NSCLC subjects on 31C6 are 3L+

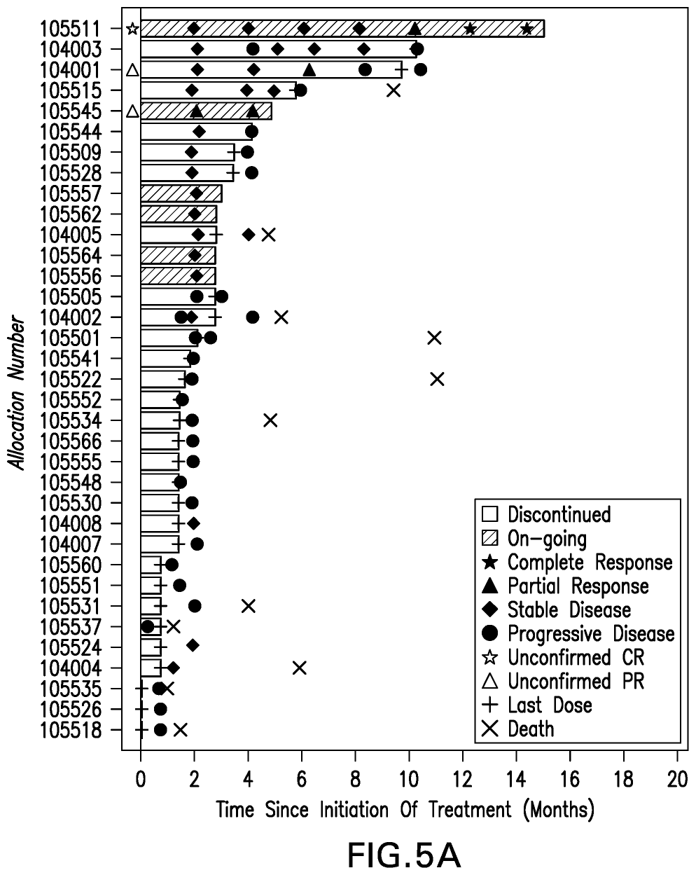

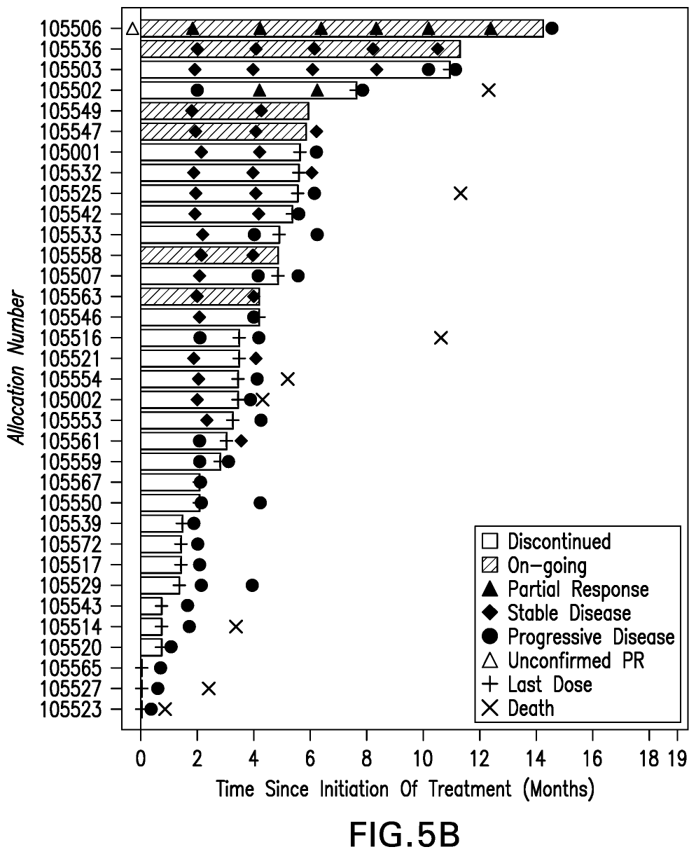

Response duration data in subjects with PD-1 refractory NSCLC treated with 31C6 (200 mg) alone or in a combination therapy with pembrolizumab (200 mg) is shown in Table 13 and FIGS. 5A and 5B.

TABLE 13
Response Duration in subjects with PD-1 Refractory
NSCLC treated with 31C6 (200 mg) + Pembrolizumab

| | | 31C6 + |
|---|---|---|
| | 31C6 | Pembrolizumab |
| Duration of Response | (N = 41) | (N = 37) |
| Number of subjects | 2 | 1 |
| with response^{†} | | |
| Time to Response | | |
| (months) | | |
| Mean | 6.1(5.8) | 1.8( ) |
| (SD) | | |
| Median (Range) | 6.1(2.1-10.2) | 1.8(1.8-1.8) |
| Response Duration^{‡} | | |
| (months) | | |
| Median (Range) | NR(2.1+-4.2+) | 12.7(12.7-12.7) |
| Number (%^{‡}) of | | |
| Subjects with Extended | | |
| Response Duration: | | |
| ≥3 months | 1(100.0) | 1(100.0) |
| ≥6 months | | 1(100.0) |

Includes subjects with confirmed complete response or partial response. From product-limit (Kaplan-Meier) method for censored data.
“+” indicates there is no progressive disease by the time of last disease assessment.
NR = Not Reached.
Database Cutoff Date: 7 May 2019

### Ovarian Cancer

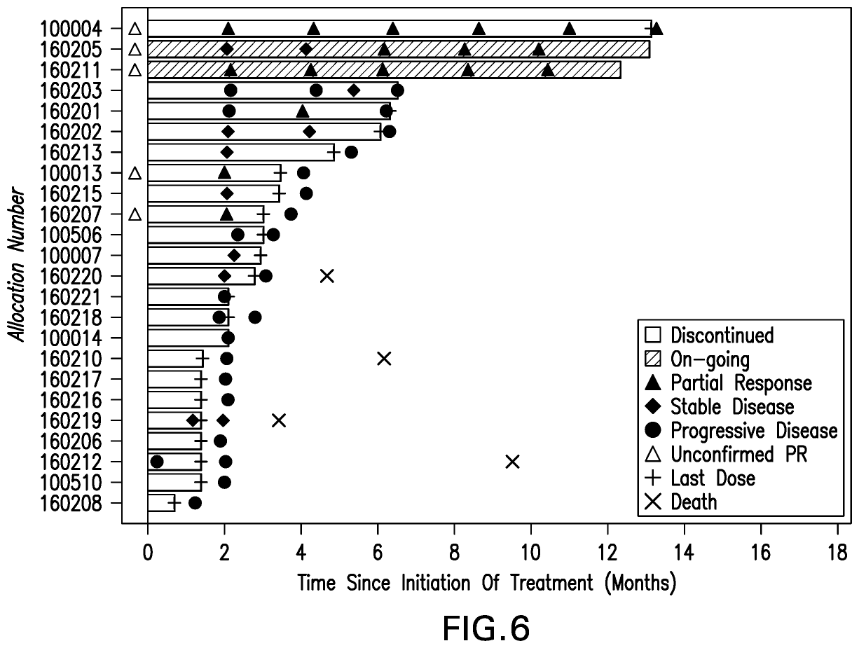

An overview of the efficacy data for the ovarian cancer patient group (part A and part B) is shown in FIG. 6 and Table 14. In the PD1 naïve ovarian cancer (resistant to platinum-based therapy) subject group, it was observed that the ORR was 19% (n=27). Additional enrichment was observed in PD-L1 positive tumors (CPS1, ORR 40%). The mean time to response was 3.5+/−2.3 months and the median DOR was 4.1-11.2+ months.

TABLE 14
Best Overall Response by RECIST1.1. by Investigator
for Ovarian cancer patients

| | Without | With |
|---|---|---|
| | confirmation | confirmation |
| Subjects in | 27 | 27 |
| population | | |
| CR | 0 (0%) | 0 (0%) |
| PR | 5 (19%) | 3 (11%) |
| ORR | 5 (19%) | 3 (11%) |
| SD | 6 (22%) | 8 (30%) |
| DCR | 11 (41%) | 11(41%) |
| PD | 13 (48%) | 13 (48%) |
| NE or NA | 3 (10%) | 3 (10%) |

Pembro + 31C6: (74% of subjects are 3L+); the three confirmed responders are 3L+ all responders BRCA negative.

Preliminary efficacy was tested in a cohort of platinum resistant ovarian cancer patients in Part B (n=20) after the observation of 2 objective responses in ovarian cancer subjects (out of 7 total) in dose escalation. In subjects with ovarian cancer treated with a combination therapy of 31C6 antibody and pembrolizumab (Parts A and B), the ORR was 19% (5/27) with a DOR of 4.2-11.4+ months. Of the 27 ovarian patients, the PD-L1 data was available for 14 subjects. In the PD-L1 positive (CPS1) tumors from ovarian patients treated with 31C6 antibody, the ORR was observed to be 40% (2/5) compared to 14% in PD-L1+ ovarian cancer subjects treated with pembrolizumab monotherapy. Notably, all ovarian responders in this study were BRCA negative. Thus, based on efficacy and tolerability, but without being limited by any specific therapy or mechanism, it is possible that the 31C6 antibody monotherapy and 31C6 antibody and pembrolizumab combination therapy could represent a very attractive chemo-free treatment option in this population of high unmet need.

### Breast Cancer

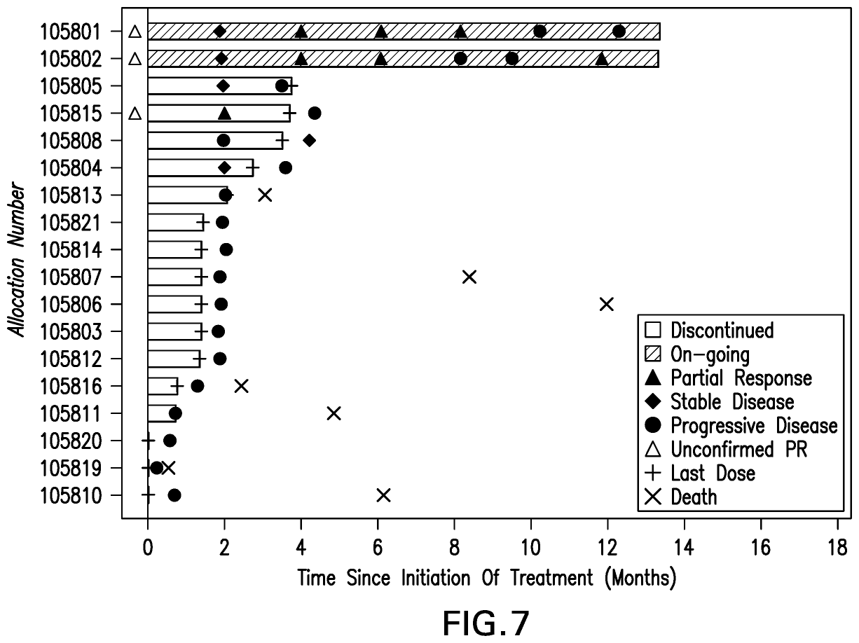

For this study there were no first line patients, such that all 2L+, PD-1/L-1 experienced patients were permitted to enroll. Of those patients, the following characteristics were identified: 1 DLT (febrile neutropenia) and 7 out of 10 patients remain on study. The discontinuation patients had the following characteristics were: 1 patient clinical PD, 2 patients had AE [PNA (not related) and G3 LFT elevation (related to io (i.e., 31C6 antibody and pembrolizumab)+pemetrexed)]. In this study 10 patients on study have had scans, all have had 1 scan only (7 SD, 2 PR, 1 PD). Data for this breast cancer study are shown in FIG. 7 and Table 15.

TABLE 15
Data for PD-1 Naïve Breast patients
treated with 31C6 (200 mg) and pembrolizumab

| | 31C6 200 mg + |
|---|---|
| | Pembrolizumab |
| Duration of Response | (N = 42) |
| Number of subjects | 2 |
| with response^{†} | |
| Time to Response | |
| (months) | |
| Mean (SD) | 3.1(1.4) |
| Median (Range) | 3.1(2.1-4.1) |
| Response Duration^{‡} | |
| (months) | |
| Median (Range) | NR (8.4+-10.4+) |
| Number (%^{‡}) of | |
| Subjects with Extended | |
| Response Duration: | |
| ≥3 months | 2(100.0) |
| ≥6 months | 2(100.0) |

Database Cutoff Date: 7 May 2019

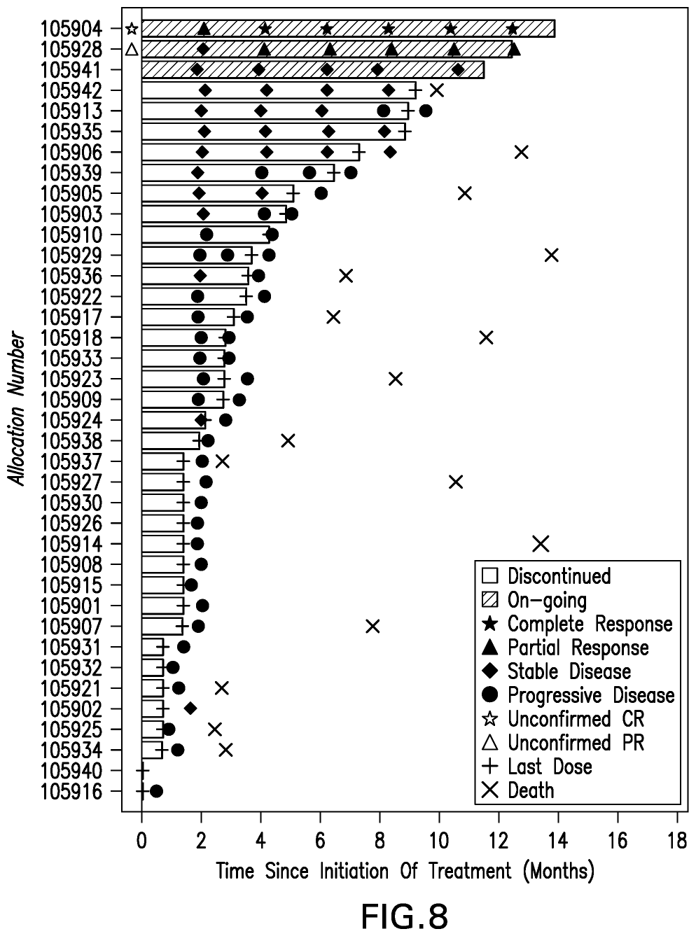

The accompanying data for CRC breast patients in FIG. 8 and Table 16.

TABLE 16
Data for PD-1 naïve CRC breast cancer patients
treated with 31C6 (200 mg) and pembrolizumab

| | 31C6 200 mg + |
|---|---|
| | Pembrolizumab |
| Duration of Response | (N = 42) |
| Number of subjects | 2 |
| with response^{†} | |
| Time to Response | |
| (months) | |
| Mean (SD) | 3.1(1.4) |
| Median (Range) | 3.1(2.1-4.1) |
| Response Duration^{‡} | |
| (months) | |
| Median (Range) | NR(8.4+-10.4+) |
| Number (%^{‡}) of | |
| Subjects with Extended | |
| Response Duration: | |
| ≥3 months | 2(100.0) |
| ≥6 months | 2(100.0) |

Database Cutoff Date: 7 May 2019

### Cervical Cancer

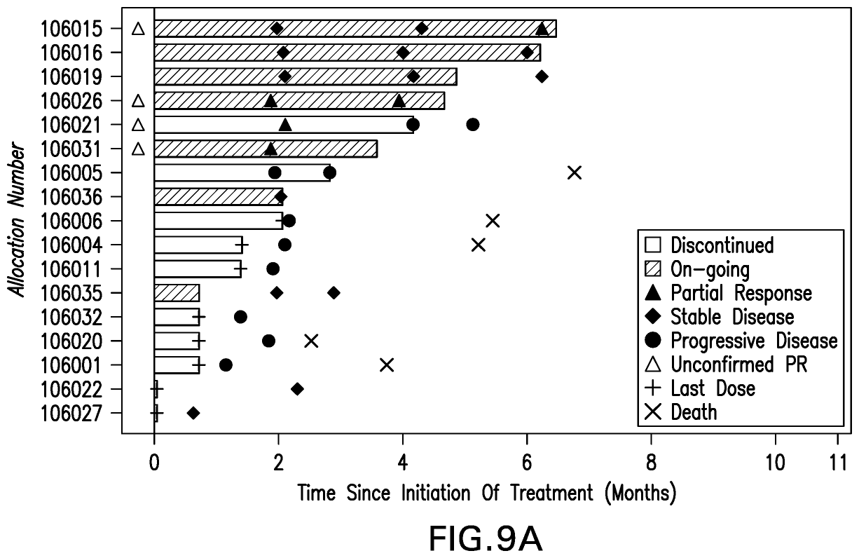

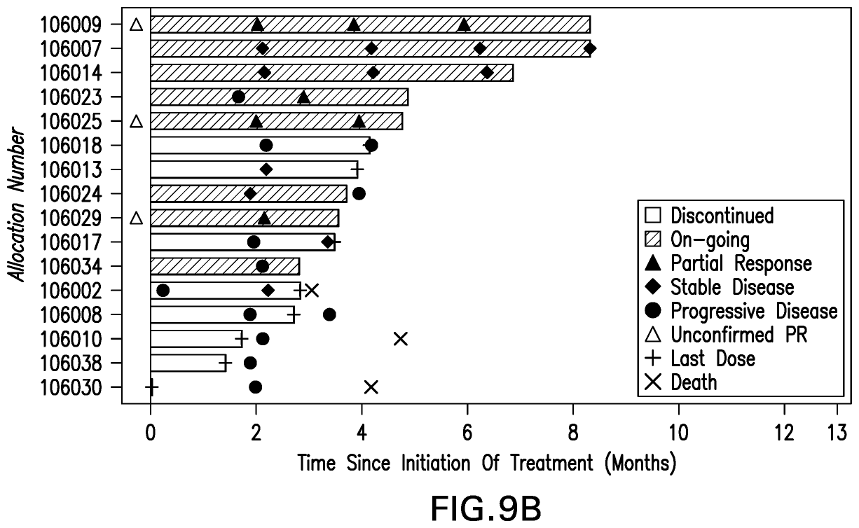

At the time of data cutoff, 7 and 8 patients remain on study at 200 mg and 700 mg doses, respectively. Data is shown in FIGS. 9A and 9B and Table 17. At this early time point, no major differences in efficacy or safety between the 200 mg and 700 mg 31C6 combination treatment arms with pembrolizumab.

TABLE 17
Data for cervical cancer patients randomized to
200 mg or 700 mg 31C6 antibody and pembrolizumab

| | 31C6 200 mg + | 31C6 700 mg + |
|---|---|---|
| | Pembro | Pembro |
| Subjects in | 18 | 19 |
| population | | |
| CR | 0 (0%) | 0 (0%) |
| PR | 4 (22%) | 3 (16%) |
| ORR | 4 (22%) | 3 (16%) |
| SD | 6 (33%) | 4 (21%) |
| DCR | 10 (56%) | 7 (37%) |
| PD | 7 (39%) | 9 (47%) |
| NE or NA | 1 (6%) | 3 (16%) |

In conclusion, it was observed that antibody 31C6 monotherapy was well tolerated. Any side effects were tolerable and the side effect profile observed for the 31C6 antibody in the combination with pembrolizumab was similar to results observed for pembrolizumab monotherapy. In terms of dose and PK data, it was observed that the half-life of 31C6 antibody was approximately 10 days. Normal PK variability was observed, and the ADA incidence observed was 10-20%. While many of the doses showed favorable results, the preliminary RP2D of 200 mg remains favorable. Data show that the exposure of the antibody treatment maintained throughout the dosing interval. Initial data show comparable safety/efficacy for 200 mg and 700 mg 31C6 antibody in randomized dose comparison Amongst the favorable efficacy data, it was observed that there were significant signals of clinical efficacy for treating NSCLC (PD-1 Naïve and PD-1 refractory) patients and ovarian cancer patients with 31C6 antibody as a monotherapy and also with a combination therapy with both 31C6 antibody and pembrolizumab. Most importantly, there was evidence that treatment with 31C6 antibody (both as part of a monotherapy therapy and a combination therapy) had antitumor activity in each of the cancers tested. For example, monotherapy antitumor activity was observed in PD-1 refractory NSCLC patients. Data included a CR in a patient treated with 31C6 monotherapy chain and 113 in the heavy chain.

### REFERENCES

1. Sharpe, A. H, Wherry, E. J., Ahmed R., and Freeman G. J. The function of programmed cell death 1 and its ligands in regulating autoimmunity and infection. Nature Immunology (2007); 8:239-245.
2. Dong H et al. Tumor-associated B7-H1 promotes T-cell apoptosis: a potential mechanism of immune evasion. Nat Med. 2002 August; 8(8):793-800.
3. Yang et al. PD-1 interaction contributes to the functional suppression of T-cell responses to human melanoma cells in vitro. Invest Ophthalmol Vis Sci. 2008 June; 49(6 (2008): 49: 2518-2525.
4. Ghebeh et al. The B7-H1 (PD-L1) T lymphocyte-inhibitory molecule is expressed in breast cancer patients with infiltrating ductal carcinoma: correlation with important high-risk prognostic factors. Neoplasia (2006) 8: 190-198.
5. Hamanishi J et al. Programmed cell death 1 ligand 1 and tumor-infiltrating CD8+ T lymphocytes are prognostic factors of human ovarian cancer. Proceeding of the National Academy of Sciences (2007): 104: 3360-3365.
6. Thompson R H et al. Significance of B7-H1 overexpression in kidney cancer. Clinical genitourin Cancer (2006): 5: 206-211.
7. Nomi, T. Sho, M., Akahori, T., et al. Clinical significance and therapeutic potential of the programmed death-1 ligand/programmed death-1 pathway in human pancreatic cancer. Clinical Cancer Research (2007); 13:2151-2157.
8. Ohigashi Y et al. Clinical significance of programmed death-1 ligand-1 and programmed death-1 ligand 2 expression in human esophageal cancer. Clin. Cancer Research (2005): 11: 2947-2953.
9. Inman et al. PD-L1 (B7-H1) expression by urothelial carcinoma of the bladder and BCG-induced granulomata: associations with localized stage progression. Cancer (2007): 109: 1499-1505.
10. Shimauchi T et al. Augmented expression of programmed death-1 in both neoplasmatic and nonneoplastic CD4+ T-cells in adult T-cell Leukemia/Lymphoma. Int. J. Cancer (2007): 121:2585-2590.
11. Gao et al. Overexpression of PD-L1 significantly associates with tumor aggressiveness and postoperative recurrence in human hepatocellular carcinoma. Clinical Cancer Research (2009) 15: 971-979.
12. Nakanishi J. Overexpression of B7-H1 (PD-L1) significantly associates with tumor grade and postoperative prognosis in human urothelial cancers. Cancer Immunol Immunother. (2007) 56:1173-1182.
13. Hino et al. Tumor cell expression of programmed cell death-1 is a prognostic factor for malignant melanoma. Cancer (2010): 00: 1-9.
14. Ghebeh H. Foxp3+ tregs and B7-H1+/PD-1+ T lymphocytes co-infiltrate the tumor tissues of high-risk breast cancer patients: implication for immunotherapy. BMC Cancer. 2008 Feb. 23; 8:57.
15. Ahmadzadeh M et al. Tumor antigen-specific CD8 T cells infiltrating the tumor express high levels of PD-1 and are functionally impaired. Blood (2009) 114: 1537-1544.
16. Thompson R H et al. PD-1 is expressed by tumor infiltrating cells and is associated with poor outcome for patients with renal carcinoma. Clinical Cancer Research (2007) 15: 1757-1761.

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents. To the extent that the references provide a definition for a claimed term that conflicts with the definitions provided in the instant specification, the definitions provided in the instant specification shall be used to interpret the claimed invention.

1. A method for treating cancer in a patient comprising administering to the patient 2.1 mg to 700 mg of an anti-TIGIT antibody comprising a heavy chain and a light chain, wherein the light chain comprises light chain CDRs of SEQ ID NOs: 26, 27 and 28 and the heavy chain comprises heavy chain CDRs of SEQ ID NOs: 29, 30 and 31.

2. The method of claim 1, wherein the anti-TIGIT antibody is administered via intravenous infusion.

3. The method of claim 1, wherein the patient is administered 2.1 mg, 7 mg, 21 mg, 70 mg, 200 mg, 210 mg, or 700 mg of the anti-TIGIT antibody.

4. (canceled)

5. (canceled)

6. (canceled)

7. (canceled)

8. (canceled)

9. (canceled)

10. The method of claim 1, wherein the patient is administered the anti-TIGIT antibody on Day 1 and then once every three weeks thereafter.

11. The method of claim 1, wherein the anti-TIGIT antibody comprises heavy chain and a light chain, and wherein the heavy chain comprises a heavy chain variable region comprising SEQ ID NO:25 and the light chain comprises a light chain variable region comprising SEQ ID NO: 24.

12. The method of claim 1, wherein the anti-TIGIT antibody comprises a heavy chain and a light chain, and wherein the heavy chain comprises SEQ ID NO:23 and the light chain comprises SEQ ID NO:22.

13. The method of claim 1, wherein the anti-TIGIT antibody is a 31C6 variant.

14. (canceled)

15. The method of claim 1, wherein the anti-TIGIT antibody is co-administered or co-formulated with an anti-PD-1 antibody or anti-PD-L1 antibody or antigen binding fragment thereof.

16. The method of claim 15, wherein the anti-PD-1 antibody, or antigen binding fragment thereof, specifically binds to human PD-1 and blocks the binding of human PD-L1 to human PD-1.

17. The method of claim 16, wherein the anti-PD-1 antibody, or antigen binding fragment thereof, also blocks binding of human PD-L2 to human PD-1.

18. The method of claim 17, wherein the anti-PD-1 antibody, or antigen binding fragment thereof comprises: (a) light chain CDRs of SEQ ID NOs: 1, 2 and 3 and (b) heavy chain CDRs of SEQ ID NOs: 6, 7 and 8.

19. The method of claim 18, wherein the anti-PD-1 antibody comprises a heavy chain and a light chain, and wherein the heavy chain comprises a heavy chain variable region comprising SEQ ID NO:9 and the light chain comprises a light chain variable region comprising SEQ ID NO: 4.

20. The method of claim 18, wherein the anti-PD-1 antibody comprises a heavy chain and a light chain, and wherein the heavy chain comprises a heavy chain variable region comprising SEQ ID NO: 10 and the light chain comprises a light chain variable region comprising SEQ ID NO: 5.

21. The method of claim 17, wherein the anti-PD-1 antibody is pembrolizumab or a pembrolizumab variant.

22. (canceled)

23. The method of claim 15, wherein the anti-PD-1 antibody is nivolumab, atezolizumab, durvalumab, or avelumab.

24. (canceled)

25. The method of claim 21, wherein the anti-PD-1 antibody is administered at 200 mg via intravenous infusion on Day 1 and then once every three weeks thereafter.

26. The method of claim 21, wherein the anti-PD-1 antibody is administered at 400 mg via intravenous infusion on Day 1 and then once every six weeks thereafter.

27. The method of claim 15, wherein the anti-PD-1 antibody is a humanized anti-PD-1 antibody that comprises a heavy chain and a light chain, and wherein the heavy chain comprises a heavy chain variable region comprising heavy chain CDRs of SEQ ID NOs: 6, 7 and 8 and the light chain comprises a light chain variable region comprising light chain CDRs of SEQ ID NOs: 1,2 and 3; and the anti-TIGIT antibody is a humanized anti-TIGIT antibody which comprises a heavy chain and a light chain, and wherein the heavy chain comprises a heavy chain variable region comprising heavy chain CDRs of SEQ ID NOs: 29, 30 and 31 and the light chain comprises a light chain variable region comprising light chain CDRs of SEQ ID NOs: 26, 27 and 28.

28. The method of claim 15, wherein the anti-PD-1 antibody comprises a heavy chain and a light chain, and wherein the heavy chain comprises a heavy chain variable region comprising SEQ ID NO:9 and the light chain comprises a light chain variable region comprising SEQ ID NO: 4; and the anti-TIGIT antibody comprises a heavy chain and a light chain, and wherein the heavy chain comprises a heavy chain variable region comprising SEQ ID NO:25 and the light chain comprises a light chain variable region comprising SEQ ID NO: 24.

29. The method of claim 15, wherein the anti-PD-1 antibody comprises a heavy chain and a light chain, and wherein the heavy chain comprises SEQ ID NO:10 and the light chain comprises SEQ ID NO: 5; and the anti-TIGIT antibody comprises a heavy chain and a light chain, and wherein the heavy chain comprises SEQ ID NO:23 and the light chain comprises a light chain variable region comprising SEQ ID NO: 22.

30. The method of claim 29, wherein the anti-PD-1 antibody is administered at 200 mg via intravenous infusion on Day 1 and then once every three weeks thereafter, and the anti-TIGIT antibody is administered at 200 mg via intravenous infusion on Day 1 and then once every three weeks thereafter.

31. The method of claim 29, wherein the anti-PD-1 antibody is administered at 400 mg via intravenous infusion on Day 1 and then once every six weeks thereafter, and the anti-TIGIT antibody is administered at 200 mg via intravenous infusion on Day 1 once every three weeks.

32. The method of claim 29, wherein the anti-PD-1 antibody is administered at 200 mg via intravenous infusion on Day 1 and then once every three weeks thereafter, and the anti-TIGIT antibody is administered at 700 mg via intravenous infusion on Day 1 and then once every three weeks thereafter.

33. The method of claim 29, wherein the anti-PD-1 antibody is administered at 400 mg via intravenous infusion on Day 1 and then once every six weeks thereafter, and the anti-TIGIT antibody is administered at 700 mg via intravenous infusion on Day 1 once every three weeks.

34. (canceled)

35. (canceled)

36. The method of claim 1, wherein the cancer is selected from the group consisting of: NSCLC, cervical cancer, colorectal cancer, hematologic cancer, gastric cancer, breast cancer, ovarian cancer, epithelial cancer, fallopian tube cancer, or primary peritoneal carcinoma.

37. The method of claim 36, wherein the cancer is NSCLC.

38. The method of claim 1, the method further comprising administering a combination of carboplatin and pemetrexed or (ii) carboplatin and paclitaxel.

39. The method of claim 1, wherein the individual has not been previously treated with anti-PD-1 or anti-PD-L1 therapy or is confirmed progressive while receiving prior anti-PD-1 or anti-PD-L1 therapy.

40. (canceled)

41. A pharmaceutical composition comprising 200 mg or 400 mg pembrolizumab or a pembrolizumab variant, 2.1 to 700 mg of 31C6 antibody or a 31C6 variant, wherein the 31C6 antibody or 31C6 variant comprises a heavy chain and a light chain, wherein the light chain comprises light chain CDRs of SEQ ID NOs: 26, 27 and 28 and the heavy chain comprises heavy chain CDRs of SEQ ID NOs: 29, 30 and 31,
and a pharmaceutically acceptable excipient.

42. (canceled)

43. The pharmaceutical composition of claim 41 comprising 2.1 mg, 7 mg, 21 mg, 70 mg, 200 mg, 210 mg, or 700 mg of the 31C6 antibody or 31C6 variant.

44. (canceled)

45. (canceled)

46. (canceled)

47. (canceled)

48. (canceled)

49. (canceled)

50. (canceled)

51. The pharmaceutical composition of claim 41, wherein the 31C6 antibody comprises a heavy chain and a light chain, wherein the heavy chain comprises a heavy chain variable region comprising SEQ ID NO:25 and the light chain comprises a light chain variable region comprising SEQ ID NO: 24.

52. The pharmaceutical composition of claim 41, wherein the 31C6 antibody comprises a heavy chain and a light chain, and wherein the heavy chain comprises SEQ ID NO:23 and the light chain comprises a light chain variable region comprising SEQ ID NO: 22.

53. A kit for treating cancer comprising any of the pharmaceutical compositions of claim 41, and instructions for use.


SEQUENCE LISTING (33 sequences)

SEQ ID NO: 1 - protein, 15 aa, Artificial Sequence
  Synthetic Polypeptide
RASKGVSTSGYSYLH

SEQ ID NO: 2 - protein, 7 aa, Artificial Sequence
  Synthetic Polypeptide
LASYLES

SEQ ID NO: 3 - protein, 9 aa, Artificial Sequence
  Synthetic Polypeptide
QHSRDLPLT

SEQ ID NO: 4 - protein, 111 aa, Artificial Sequence [sequence omitted]

SEQ ID NO: 5 - protein, 218 aa, Artificial Sequence
  Synthetic Polypeptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 5 aa, Artificial Sequence
  Synthetic Polypeptide
NYYMY

SEQ ID NO: 7 - protein, 17 aa, Artificial Sequence
  Synthetic Polypeptide
GINPSNGGTNFNEKFKN

SEQ ID NO: 8 - protein, 11 aa, Artificial Sequence
  Synthetic Polypeptide
RDYRFDMGFDY

SEQ ID NO: 9 - protein, 120 aa, Artificial Sequence [sequence omitted]

SEQ ID NO: 10 - protein, 447 aa, Artificial Sequence
  Synthetic Polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, Artificial Sequence
  Synthetic Polypeptide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 440 aa, Artificial Sequence
  Synthetic Polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 17 aa, Artificial Sequence
  Synthetic Polypeptide
KSSQSLLHTSTRKNYLA

SEQ ID NO: 14 - protein, 7 aa, Artificial Sequence
  Synthetic Polypeptide
WASTRES

SEQ ID NO: 15 - protein, 8 aa, Artificial Sequence
  Synthetic Polypeptide
KQSYDVVT

SEQ ID NO: 16 - protein, 112 aa, Artificial Sequence
  Synthetic Polypeptide
DIVMSQSPSSLAVSAGEKVTMTCKSSQSLLHTSTRKNYLAWYQQKPGQSPKLLIYWASTRESGVPDRFTGSGSGTDFTLTISSVQAEDLAVYYCKQSYDVVTFGAGTKLELK

SEQ ID NO: 17 - protein, 5 aa, Artificial Sequence
  Synthetic Polypeptide
SYWIH

SEQ ID NO: 18 - protein, 10 aa, Artificial Sequence
  Synthetic Polypeptide
GYTFTSYWIH

SEQ ID NO: 19 - protein, 17 aa, Artificial Sequence
  Synthetic Polypeptide
YINPSSGYHEYNQKFID

SEQ ID NO: 20 - protein, 13 aa, Artificial Sequence
  Synthetic Polypeptide
SGWLIHGDYYFDF

SEQ ID NO: 21 - protein, 122 aa, Artificial Sequence
  Synthetic Polypeptide
  MISC_FEATURE (1)..(1): x=Q or pE (pyro-glutamate)
XVHLQQSGAELAKPGASVKMSCKASGYTFTSYWIHWIKQRPGQGLEWIGYINPSSGYHEYNQKFIDKATLTADRSSSTAYMHLTSLTSEDSAVYYCARSGWLIHGDYYFDFWGQGTTLTVSS

SEQ ID NO: 22 - protein, 214 aa, Artificial Sequence
  Synthetic Polypeptide
DIQMTQSPSSLSASVGDRVTITCRASEHIYSYLSWYQQKPGKVPKLLIYNAKTLAEGVPSRFSGSGSGTDFTLTISSLQPEDVATYYCQHHFGSPLTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 449 aa, Artificial Sequence
  Synthetic Polypeptide
EVQLVQSGAEVKKPGSSVKVSCKASGYTFSSYVMHWVRQAPGQGLEWIGYIDPYNDGAKYAQKFQGRVTLTSDKSTSTAYMELSSLRSEDTAVYYCARGGPYGWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 107 aa, Artificial Sequence
  Synthetic Polypeptide
DIQMTQSPSSLSASVGDRVTITCRASEHIYSYLSWYQQKPGKVPKLLIYNAKTLAEGVPSRFSGSGSGTDFTLTISSLQPEDVATYYCQHHFGSPLTFGQGTRLEIK

SEQ ID NO: 25 - protein, 119 aa, Artificial Sequence
  Synthetic Polypeptide
EVQLVQSGAEVKKPGSSVKVSCKASGYTFSSYVMHWVRQAPGQGLEWIGYIDPYNDGAKYAQKFQGRVTLTSDKSTSTAYMELSSLRSEDTAVYYCARGGPYGWYFDVWGQGTTVTVSS

SEQ ID NO: 26 - protein, 11 aa, Artificial Sequence
  Synthetic Polypeptide
RASEHIYSYLS

SEQ ID NO: 27 - protein, 7 aa, Artificial Sequence
  Synthetic Polypeptide
NAKTLAE

SEQ ID NO: 28 - protein, 9 aa, Artificial Sequence
  Synthetic Polypeptide
QHHFGSPLT

SEQ ID NO: 29 - protein, 5 aa, Artificial Sequence
  Synthetic Polypeptide
SYVMH

SEQ ID NO: 30 - protein, 17 aa, Artificial Sequence
  Synthetic Polypeptide
YIDPYNDGAKYAQKFQG

SEQ ID NO: 31 - protein, 10 aa, Artificial Sequence
  Synthetic Polypeptide
GGPYGWYFDV

SEQ ID NO: 32 - protein, 290 aa, Artificial Sequence
  Synthetic Polypeptide
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 33 - protein, 244 aa, Artificial Sequence
  Synthetic Polypeptide
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG